(19) United States
(12) Patent Application Publication
(10) Pub. No.: US 2015/0314034 A1
(43) Pub. Date: Nov. 5, 2015

(54) PROCESS FOR PRODUCING WATER-ABSORBING POLYMER PARTICLES

(71) Applicant: BASF SE, Ludwigshafen (DE)

(72) Inventors: Klaus Dieter Hörner, Lampertheim (DE); Markus Linsenbühler, Heddesheim (DE); Bernd Siegel, Otterstadt (DE)

(73) Assignee: BASF SE, Ludwigshafen (DE)

(21) Appl. No.: 14/651,712

(22) PCT Filed: Dec. 13, 2013
(86) PCT No.: PCT/EP2013/076545

§ 371 Date: Jun. 12, 2015

Related U.S. Application Data
(60) Provisional application No. 61/740,473, filed on Dec. 21, 2012.

(30) Foreign Application Priority Data
Dec. 21, 2012 (EP) 12199122.8

(51) Int. Cl. A61L 15/60; B05D 1/22; A61L 15/22; B05D 1/02

(52) U.S. Cl. CPC A61L 15/60; B05D 1/02; B05D 1/22; A61L 15/225

(57) ABSTRACT

A process for preparing water-absorbing polymer particles, comprising polymerisation of a foamed monomer solution or suspension, drying, grinding, classification and spray-coating the water-absorbing polymeric particles with an elastic film-forming polymer in a fluidised bed reactor in the range from 0° C. to 50° C. and heat-treating the coated particles at a temperature above 0° C.

The present invention relates to a process for preparing water-absorbing polymer particles, comprising polymerisation of a foamed monomer solution or suspension, drying, grinding and classification.

Being products which absorb aqueous solutions, water-absorbing polymers are used to produce diapers, tampons, sanitary napkins, panty liners, wound dressings and other hygiene articles, but also as water-retaining agents in market gardening.

The production of water-absorbing polymer particles is described in the monograph “Modern Superabsorbent Polymer Technology”, F. L. Buchholz and A. T. Graham, Wiley-VCH, 1998, pages 71 to 103.

Water-absorbing foams based on crosslinked monomers comprising acid groups are known, for example from EP 0 858 478 B1, WO 97/31971 A1, WO 99/44648 A1 and WO 00/52087 A1. They are produced, for example, by foaming a polymerisable aqueous mixture which comprises at least 50 mol % of neutralised, ethylenically unsaturated monomers comprising acid groups, crosslinker and at least one surfactant, and then polymerizing the foamed mixture. The polymerisable mixture can be foamed by dispersing fine bubbles of a gas which is inert toward free radicals or by dissolving such a gas under elevated pressure in the polymerisable mixture and decompressing the mixture. The foams are used, for example, in hygiene articles for acquisition, distribution and storage of body fluids. WO 2011/061 315 A1 discloses comminuted foams that exhibit high permeability for liquids and a high swelling speed. WO 2006/082 239 A2 relates to a process for producing water-absorbing particles comprising coating the water-absorbing polymeric particles with an elastic film-forming polymer in a fluidised bed reactor in the range from 0° C. to 50° C. and heat-treating the coated particles at a temperature above 50° C.

It was an object of the present invention to provide water-absorbing polymer particles with an improved profile of properties, such as high saline flow conductivity (SFC) and especially a high free swell rate (FSR).

The object was achieved by a process for producing water-absorbing polymer particles by polymerizing a foamed aqueous monomer solution or suspension comprising
a) at least one ethylenically unsaturated monomer which bears acid groups and has been neutralised to an extent of 25 to 95 mol %,
b) at least one crosslinker,
c) at least one initiator and
d) optionally at least one surfactant,
e) optionally one or more ethylenically unsaturated monomers copolymerisable with the monomers mentioned under a),
f) optionally a solubiliser and
g) optionally thickeners, foam stabilisers, polymerisation regulators, fillers, fibres and/or cell nucleators,
the monomer solution or suspension being polymerised to a polymeric foam that is dried and subsequently ground and classified, the process further comprising
i) spray-coating water-absorbing polymeric particles with an elastic film-forming polymer in a fluidised bed reactor in the range from 0° C. to 50° C. and
ii) heat-treating the coated particles at a temperature above 50° C.

In preferred embodiment, the process of the invention comprises:
i) spray-coating water-absorbing polymeric particles with an elastic film-forming polymer in a fluidised bed reactor, preferably in a continuous process, in the range from 0° C. to 50° C., preferably to less than 45° C., and
ii) heat-treating the coated particles at a temperature above 50° C.

Further, a water-absorbing material has been found that is obtainable by the process of this invention. The water-absorbing polymer particles obtained are typically water-insoluble. Further, hygiene articles have been found that comprise the water-absorbing material of the invention.

The monomers a) are preferably water-soluble, i.e. the solubility in water at 23° C. is typically at least 1 g/100 g of water, preferably at least 5 g/100 g of water, more preferably at least 25 g/100 g of water, most preferably at least 35 g/100 g of water.

Suitable monomers a) are, for example, ethylenically unsaturated carboxylic acids, such as acrylic acid, methacrylic acid and itaconic acid. Particularly preferred monomers are acrylic acid and methacrylic acid. Very particular preference is given to acrylic acid.

Further suitable monomers a) are, for example, ethylenically unsaturated sulphonic acids, such as styrene sulphonic acid and 2-acrylamido-2-methylpropanesulphonic acid (AMPS).

Impurities can have a considerable influence on the polymerisation. The raw materials used should therefore have a maximum purity. It is therefore often advantageous to specially purify the monomers a). Suitable purification processes are described, for example, in WO 2002/055469 A1, WO 2003/078378 A1 and WO 2004/035514 A1. A suitable monomer a) is, for example, an acrylic acid purified according to WO 2004/035514 A1 comprising 99.8460% by weight of acrylic acid, 0.0950% by weight of acetic acid, 0.0332% by weight of water, 0.0203% by weight of propionic acid, 0.0001% by weight of furfurals, 0.0001% by weight of maleic anhydride, 0.0003% by weight of diacrylic acid and 0.0050% by weight of hydroquinone monomethyl ether.

The amount of monomer a) is preferably 20 to 90% by weight, more preferably 30 to 85% by weight, most preferably 35 to 75% by weight, based in each case on the unneutralised monomer a) and on the monomer solution or suspension. Based on the unneutralised monomer a) means in the context of this invention that the proportion of the monomer a) before the neutralisation is used for the calculation, i.e. the contribution of the neutralisation is not taken into account.

The acid groups of the monomers a) have been neutralised to an extent of 25 to 95 mol %, preferably to an extent of 40 to 85 mol %, more preferably to an extent of 50 to 80 mol %, especially preferably to an extent of 55 to 75 mol %, for which the customary neutralizing agents can be used, for example alkali metal hydroxides, alkali metal oxides, alkali metal carbonates or alkali metal hydrogen carbonates, and mixtures thereof. The neutralisation can, however, also be undertaken with ammonia, amines or alkanolamines, such as ethanolamine, diethanol amine or triethanol amine.

In a preferred embodiment of the present invention, at least 50 mol %, preferably at least 75 mol %, more preferably at least 90 mol %, most preferably at least 95 mol %, of the neutralised monomers a) have been neutralised by means of an inorganic base, preferably potassium carbonate, sodium carbonate or sodium hydroxide.

A high degree of neutralisation and a high proportion of acid groups neutralised with an inorganic base reduce the flexibility of the polymeric foams obtained and ease the subsequent grinding.

The proportion of acrylic acid and/or salts thereof in the total amount of monomers a) is preferably at least 50 mol %, more preferably at least 90 mol %, most preferably at least 95 mol %.

The monomers a) typically comprise polymerisation inhibitors, preferably hydroquinone monoethers, as storage stabilisers.

The monomer solution comprises preferably up to 250 ppm by weight, preferably at most 130 ppm by weight, more preferably at most 70 ppm by weight, preferably at least 10 ppm by weight, more preferably at least 30 ppm by weight, especially around 50 ppm by weight, of hydroquinone monoether, based in each case on the unneutralised monomer a). For example, the monomer solution can be prepared by using an ethylenically unsaturated monomer bearing acid groups with an appropriate content of hydroquinone monoether.

Preferred hydroquinone monoethers are hydroquinone monomethyl ether (MEHQ) and/or alpha-tocopherol (vitamin E).

Suitable crosslinkers b) are compounds having at least two groups suitable for crosslinking. Such groups are, for example, ethylenically unsaturated groups which can be polymerised free-radically into the polymer chain, and functional groups which can form covalent bonds with the acid groups of the monomer a). In addition, polyvalent metal salts which can form coordinate bonds with at least two acid groups of the monomer a) are also suitable as crosslinkers b).

Crosslinkers b) are preferably compounds having at least two polymerisable groups which can be polymerised free-radically into the polymer network. Suitable crosslinkers b) are, for example, ethylene glycol dimethacrylate, diethylene glycol diacrylate, polyethylene glycol diacrylate, allyl methacrylate, trimethylolpropane triacrylate, triallylamine, tetraallylammonium chloride, tetraallyloxyethane, as described in EP 0 530 438 A1, di- and triacrylates, as described in EP 0 547 847 A1, EP 0 559 476 A1, EP 0 632 068 A1, WO 93/21237 A1, WO 2003/104299 A1, WO 2003/104300 A1, WO 2003/104301 A1 and DE 103 31 450 A1, mixed acrylates which, as well as acrylate groups, comprise further ethylenically unsaturated groups, as described in DE 103 31 456 A1 and DE 103 55 401 A1, or crosslinker mixtures, as described, for example, in DE 195 43 368 A1, DE 196 46 484 A1, WO 90/15830 A1 and WO 2002/032962 A2.

Preferred crosslinkers b) are pentaerythrityl triallyl ether, tetraalloxyethane, methylenebismethacrylamide, 15-tuply ethoxylated trimethylolpropane triacrylate, polyethylene glycol diacrylate, trimethylolpropane triacrylate and triallylamine.

Very particularly preferred crosslinkers b) are the polyethoxylated and/or -propoxylated glycerols which have been esterified with acrylic acid or methacrylic acid to give di- or triacrylates, as described, for example, in WO 2003/104301 A1. Di- and/or triacrylates of 3- to 10-tuply ethoxylated glycerol are particularly advantageous. Very particular preference is given to di- or triacrylates of 1- to 5-tuply ethoxylated and/or propoxylated glycerol. Most preferred are the triacrylates of 3- to 5-tuply ethoxylated and/or propoxylated glycerol, especially the triacrylate of 3-tuply ethoxylated glycerol.

The amount of crosslinker b) is preferably 1 to 10% by weight, more preferably 2 to 7% by weight and most preferably 3 to 5% by weight, based in each case on the unneutralised monomer a). With rising crosslinker content, the centrifuge retention capacity (CRC) falls and the absorption under a pressure of 21.0 g/cm² (AUL 0.3 psi) passes through a maximum.

The initiators c) may be all compounds which generate free radicals under the polymerisation conditions, for example thermal initiators, redox initiators, photo initiators.

Thermal initiators are, for example, peroxides, hydro peroxides, hydrogen peroxide, persulphates and azo initiators. Suitable azo initiators are, for example, 2,2′-azobis(2-amidinopropane) dihydrochloride, 2,2′-azobis(N,N-dimethylene)isobutyramidine dihydrochloride, 2-(carbamoylazo)isobutyronitrile, 2,2′-azobis[2-(2′-imidazolin-2-yl)propane]dihydrochloride and 4,4′-azobis(4-cyanovaleric acid).

Photo initiators are, for example, α-splitters, H-abstracting systems and azides. Suitable α-splitters or H-abstracting systems are, for example, benzophenone derivatives such as Michler's ketone, phenanthrene derivatives, fluorine derivatives, anthraquinone derivatives, thioxanthone derivatives, coumarin derivatives, benzoin ethers and derivatives thereof, azo initiators such as the abovementioned free-radical formers, substituted hexaarylbisimidazoles or acylphosphine oxides. Suitable azides are, for example, 2-(N,N-dimethylamino)ethyl 4-azidocinnamate, 2-(N,N-dimethylamino)ethyl 4-azidonaphthyl ketone, 2-(N,N-dimethylamino)ethyl 4-azidobenzoate, 5-azido-1-naphthyl 2′-(N,N-dimethylamino)ethyl sulphone, N-(4-sulphonylazidophenyl)maleimide, N-acetyl-4-sulphonylazidoaniline, 4-sulphonylazidoaniline, 4-azidoaniline, 4-azidophenacyl bromide, p-azidobenzoic acid, 2,6-bis(p-azidobenzylidene)cyclohexanone and 2,6-bis(pazidobenzyl idene)-4-methylcyclohexanone.

The initiators c) are used in customary amounts, preferably at least 0.01 mol %, more preferably at least 0.05 mol %, most preferably at least 1 mol %, and typically less than 5 mol %, preferably less than 2 mol %, based on the monomers a).

In a preferred embodiment surfactants d) are added during the preparation of the foamed monomer solution or suspension to increase its stability. It is possible to use anionic, cationic or non-ionic surfactants or surfactant mixtures which are compatible with one another. It is possible to use low molecular weight or else polymeric surfactants, combinations of different types or else the same type of surfactants having been found to be advantageous. Usable non-ionic surfactants are, for example, addition products of alkylene oxides, especially ethylene oxide, propylene oxide and/or butylene oxide, onto alcohols, amines, phenols, naphthols or carboxylic acids. The surfactants used are advantageously addition products of ethylene oxide and/or propylene oxide onto alcohols comprising at least 10 carbon atoms, where the addition products comprise 3 to 200 mol of ethylene oxide and/or propylene oxide added on per mole of alcohol. The addition products comprise the alkylene oxide units in the form of blocks or in random distribution. Examples of usable non-ionic surfactants are the addition products of 7 mol of ethylene oxide onto 1 mol of tallow fat alcohol, reaction products of 9 mol of ethylene oxide with 1 mol of tallow fat alcohol, and addition products of 80 mol of ethylene oxide onto 1 mol of tallow fat alcohol. Further usable commercial non-ionic surfactants consist of reaction products of oxo alcohols or Ziegler alcohols with 5 to 12 mol of ethylene oxide per mole of alcohol, especially with 7 mol of ethylene oxide. Further usable commercial non-ionic surfactants are obtained by ethoxylation of castor oil. For example, 12 to 80 mol of ethylene oxide are added on per mole of castor oil. Further usable commercial products are, for example, the reaction products of 18 mol of ethylene oxide with 1 mol of tallow fat alcohol, the addition products of 10 mol of ethylene oxide onto 1 mol of a C₁₃/C₁₅ oxo alcohol, or the reaction products of 7 to 8 mol of ethylene oxide onto 1 mol of a C₁₃/C₁₅ oxo alcohol. Further suitable non-ionic surfactants are phenol alkoxylates, for example p-tert.-butyl phenol which has been reacted with 9 mol of ethylene oxide, or methyl ethers of reaction products of 1 mol of a C₁₂- to C₁₈-alcohol and 7.5 mol of ethylene oxide.

The above-described non-ionic surfactants can be converted to the corresponding sulphuric monoesters, for example, by esterification with sulphuric acid. The sulphuric monoesters are used as anionic surfactants in the form of the alkali metal or ammonium salts. Suitable anionic surfactants are, for example, alkali metal or ammonium salts of sulphuric monoesters of addition products of ethylene oxide and/or propylene oxide onto fatty alcohols, alkali metal or ammonium salts of alkylbenzene sulphonic acid or of alkyl phenol ether sulphates. Products of the type mentioned are commercially available. For example, the sodium salt of a sulphuric monoester of a C₁₃/C₁₅ oxo alcohol reacted with 106 mol of ethylene oxide, the triethanolamine salt of dodecylbenzene sulphonic acid, the sodium salt of alkylphenol ether sulphates and the sodium salt of the sulphuric monoester of a reaction product of 106 mol of ethylene oxide with 1 mol of tallow fat alcohol are commercial usable anionic surfactants. Further suitable anionic surfactants are sulphuric monoesters of C₁₃/C₁₅ oxo alcohols, paraffin sulphonic acids such as 015 alkylsulphonate, alkyl-substituted benzene sulphonic acids and alkyl-substituted naphthalene sulphonic acids such as dodecylbenzene sulphonic acid and di-n-butylnaphthalene sulphonic acid, and also fatty alcohol phosphates such as C₁₅/C₁₈ fatty alcohol phosphate. The polymerisable aqueous mixture may comprise combinations of a non-ionic surfactant and an anionic surfactant, or combinations of non-ionic surfactants or combinations of anionic surfactants. Cationic surfactants are also suitable. Examples thereof are the dimethyl sulphate-quaternised reaction products of 6.5 mol of ethylene oxide with 1 mol of oleyl amine, distearyldimethylammonium chloride, lauryltrimethylammonium chloride, cetylpyridinium bromide, and dimethyl sulphate-quaternised stearic acid triethanolamine ester, which is preferably used as a cationic surfactant.

The surfactant content, based on the unneutralised monomer a) is preferably 0.01 to 10% by weight, more preferably 0.1 to 5% by weight, most preferably 0.5 to 3% by weight.

Ethylenically unsaturated monomers e) copolymerisable with the ethylenically unsaturated monomers a) bearing acid groups are, for example, acryl amide, methacryl amide, hydroxyethyl acrylate, hydroxyethyl methacrylate, dimethylaminoethyl methacrylate, dimethylaminoethyl acrylate, dimethylaminopropyl acrylate, diethylaminopropyl acrylate, dimethylaminoethyl methacrylate, diethylaminoethyl methacrylate.

Solubilisers f) are water-miscible organic solvents, for example dimethyl sulphoxide, dimethyl formamide, N-methyl pyrrolidone, monohydric alcohols, glycols, polyethylene glycols or monoethers derived therefrom, where the monoethers comprise no double bonds in the molecule. Suitable ethers are methyl glycol, butyl glycol, butyl diglycol, methyl diglycol, butyl triglycol, 3-ethoxy-1-propanol and glyceryl monomethyl ether.

If solubilisers f) are used, the content thereof in the monomer solution or suspension is preferably up to 50% by weight, more preferably 1 to 25% by weight, most preferably 5 to 10% by weight.

The monomer solution or suspension may comprise thickeners, foam stabilisers, fillers, fibres and/or cell nucleators g). Thickeners are used, for example, to optimise the foam structure and to improve the foam stability. This reduces or prevents shrinking of the foam during polymerisation. Useful thickeners include all natural and synthetic polymers which are known for this purpose. They increase the viscosity of an aqueous system significantly and do not react with the amino groups of the basic polymer. These may be water-swellable or water-soluble synthetic and natural polymers. A detailed overview of thickeners can be found, for example, in the publications by R. Y. Lochhead and W. R. Fron, Cosmetics & Toiletries, 108, 95-135 (May 1993) and M. T. Clarke, “Rheological Additives” in D. Laba (ed.) “Rheological Properties of Cosmetics and Toiletries”, Cosmetic Science and Technology Series, Vol. 13, Marcel Dekker Inc., New York 1993.

Water-swellable or water-soluble synthetic polymers useful as thickeners are, for example, high molecular weight polyethylene glycols or copolymers of ethylene glycol and propylene glycol, and high molecular weight polysaccharides such as starch, guar flour, carob flour, or derivatives of natural substances, such as carboxymethyl cellulose, hydroxyethyl cellulose, hydroxymethyl cellulose, hydroxypropyl cellulose and cellulose mixed ethers. A further group of thickeners is that of water-insoluble products such as fine silica, zeolites, bentonite, cellulose powder or other fine powders of crosslinked polymers. The monomer solution or suspension may comprise the thickeners in amounts up to 30% by weight. If such thickeners are used at all, they are present in the monomer solution or suspension in amounts of 0.1 to 10% by weight, preferably 0.5 to 20% by weight.

The water-absorbing polymer particles described herein, preferably prior to coating with film-forming polymer, may be used as thickeners. Further, conventional water-absorbing polymers (superabsorbents) that are not derived from foams as described herein may be used, in particular superabsorbent fines having a mean particle size below 150 μm.

In order to optimise the foam structure, it is optionally possible to add hydrocarbons having at least 5 carbon atoms in the molecule to the aqueous reaction mixture. Suitable hydrocarbons are, for example, pentane, cyclopentane, hexane, cyclohexane, heptane, octane, isooctane, decane and dodecane. The useful aliphatic hydrocarbons may be straight-chain, branched or cyclic and have a boiling temperature above the temperature of the aqueous mixture during the foaming. The aliphatic hydrocarbons increase the shelf life of the as yet unpolymerised foamed aqueous reaction mixture. This eases the handling of the as yet unpolymerised foams and increases process reliability. The hydrocarbons act, for example, as cell nucleators and simultaneously stabilise the foam already formed. In addition, they can bring about further foaming in the course of polymerisation of the monomer solution or suspension. They may then also have the function of a blowing agent. Instead of hydrocarbons or in a mixture therewith, it is optionally also possible to use chlorinated or fluorinated hydrocarbons as a cell nucleator and/or foam stabiliser, such as dichloromethane, trichloro methane, 1,2-dichloro ethane, trichlorofluoro methane or 1,1,2-trichlorotrifluoro ethane. If hydrocarbons are used, they are used, for example, in amounts of 0.1 to 20% by weight, preferably 0.1 to 10% by weight, based on the monomer solution or suspension.

In order to modify the properties of the foams, it is possible to add one or more fillers, for example chalk, talc, clay, titanium dioxide, magnesium oxide, aluminium oxide, precipitated silicas in hydrophilic or hydrophobic polymorphs, dolomite and/or calcium sulphate. The fillers may be present in the monomer solution or suspension in amounts of up to 30% by weight.

The above-described aqueous monomer solutions or suspensions are first foamed. It is possible, for example, to dissolve an inert gas, such as nitrogen, carbon dioxide or air, in the aqueous monomer solution or suspension under a pressure of, for example, 2 to 400 bar, and then to decompress it to standard pressure. In the course of decompression from at least one nozzle, free-flowing monomer foam forms. Since gas solubility increases with falling temperature, the gas saturation and the subsequent foaming should be performed at minimum temperature, though undesired precipitations should be avoided. It is also possible to foam the aqueous monomer solutions or suspensions by another method, by dispersing fine bubbles of an inert gas therein. In the laboratory, the aqueous monomer solutions or suspensions can be foamed, for example, by foaming the aqueous monomer solution or suspension in a food processor equipped with egg beaters. In addition, it is possible to foam the aqueous monomer solutions or suspensions with carbon dioxide, by adding carbonates or hydrogen carbonates for neutralisation.

The foam generation is preferably performed in an inert gas atmosphere and with inert gases, for example by admixing with nitrogen or noble gases under standard pressure or elevated pressure, for example up to 25 bar, and then decompressing. The consistency of the monomer foams, the size of the gas bubbles and the distribution of the gas bubbles in the monomer foam can be varied within a wide range, for example, through the selection of the surfactants d), solubilisers f), foam stabilisers, cell nucleators, thickeners and fillers g). This allows the density, the open-cell content and the wall thickness of the monomer foam to be adjusted easily. The aqueous monomer solution or suspension is preferably foamed at a temperature below the boiling point of the constituents thereof, for example at a temperature from ambient temperature up to 100° C., preferably from 0 to 50° C., more preferably from 5 to 20° C. However, it is also possible to work at temperatures above the boiling point of the component with the lowest boiling point, by foaming the aqueous monomer solution or suspension in a vessel sealed pressure-tight. This gives monomer foams which are free-flowing and stable over a prolonged period. The density of the monomer foams is, at a temperature of 20° C., for example, 0.01 to 0.9 g/cm³.

The resulting monomer foam can be polymerised on a suitable substrate. The polymerisation is performed in the presence of customary free-radical-forming initiators c). The free radicals can be generated, for example, by heating (thermal polymerisation) or by irradiation with light of a suitable wavelength (UV polymerisation).

Polymeric foams with a layer thickness of up to about 5 millimetres are produced, for example, by heating on one side or both sides, or more particularly by irradiating the monomer foams on one side or both sides. If relatively thick polymeric foams are to be produced, for example polymeric foams with thicknesses of several centimetres, heating of the monomer foam with the aid of microwaves is particularly advantageous, because relatively homogeneous heating can be achieved in this way. With increasing layer thickness, however, the proportion of unconverted monomer a) and crosslinker b) in the resulting polymeric foam increases. The thermal polymerisation is performed, for example, at temperatures of 20 to 180° C., preferably in the range from 40° C. to 160° C., especially at temperatures from 65 to 140° C. In the case of relatively thick polymeric foams, the monomer foam can be heated and/or irradiated on both sides, for example with the aid of contact heating or by irradiation or in a drying cabinet. The resulting polymeric foams are open-cell. The proportion of open cells is, for example, at least 80%, preferably above 90%. Particular preference is given to polymeric foams with an open-cell content of 100%. The proportion of open cells in the polymeric foam is determined, for example, with the aid of scanning electron microscopy.

After the polymerisation of the monomer foam or during the polymerisation, the polymeric foam is dried. In the course of this, water and other volatile constituents are removed. Examples of suitable drying processes are thermal convection drying such as forced air drying, thermal contact drying such as roller drying, radiative drying such as infrared drying, dielectric drying such as microwave drying, and freeze drying.

The optimum temperature and the optimum residence time in the dryer obviously depend on the type of dryer used, the foam thickness and whether there is any forced gas stream in the dryer, and a lower temperature can be offset by a longer residence time and vice versa. These parameters can easily be optimised by routine experiments. In typical belt dryers, the drying temperatures generally are in the range of 50 to 250° C., preferably 70 to 220° C., more preferably 80 to 210° C., most preferably 90 to 200° C. The preferred residence time at this temperature in the dryer is preferably at least 1 minute, more preferably at least 3 minutes, most preferably at least 5 minutes, and typically at most 60 minutes, more preferably at most 30 minutes, most preferably at most 10 minutes.

In order to avoid undesired decomposition and crosslinking reactions, it may be advantageous to perform the drying under reduced pressure, under a protective gas atmosphere and/or under gentle thermal conditions, under which the product temperature does not exceed 120° C., preferably 100° C. A particularly suitable drying process is (vacuum) belt drying.

After the drying step, the polymeric foam usually comprises less than 10% by weight of water. The water content of the polymeric foam can, however, be adjusted as desired by moistening with water or water vapour.

Thereafter, the dried polymeric foam is ground and classified, and can be ground typically by using one-stage or multistage roll mills, pin mills, hammer mills or vibratory mills. In a preferred embodiment of the present invention, the dried polymeric foam is first ground by means of a cutting mill and then further ground by means of a turbo mill.

Advantageously, pre-dried polymeric foam with a water content of 5 to 30% by weight, more preferably of 8 to 25% by weight, most preferably of 10 to 20% by weight, is ground and subsequently dried to the desired final water content. The grinding of merely pre-dried polymeric foam leads to fewer undesirably small polymer particles.

The water-absorbing polymer particles are screened off using appropriate screens to a particle size in the range from preferably 100 to 1 000 μm, more preferably 150 to 850 μm, most preferably of 150 to 600 μm.

The mean particle size of the polymer particles removed as the product fraction is preferably at least 200 μm, more preferably from 250 to 600 μm and very particularly from 300 to 500 μm.

The proportion of particles with a particle size of at least 150 μm is preferably at least 90% by weight, more preferably at least 95% by weight, most preferably at least 98% by weight.

Polymer particles with too small a particle size lower the permeability (SFC). The proportion of excessively small polymer particles (undersize) should therefore be small.

Excessively small polymer particles are therefore typically removed and recycled into the process. The excessively small polymer particles can be moistened with water and/or aqueous surfactant before or during the recycling.

It is also possible to remove excessively small polymer particles in later process steps, for example after the surface post-crosslinking or another coating step. In this case, the excessively small polymer particles recycled are surface post-crosslinked or coated in another way, for example with fumed silica.

The proportion of particles having a particle size of at most 850 μm is preferably at least 90% by weight, more preferably at least 95% by weight, most preferably at least 98% by weight.

The proportion of particles having a particle size of at most 710 μm is preferably at least 90% by weight, more preferably at least 95% by weight, most preferably at least 98% by weight.

The proportion of particles having a particle size of at most 600 μm is preferably at least 90% by weight, more preferably at least 95% by weight, most preferably at least 98% by weight.

Polymer particles with too great a particle size are less mechanically stable. The proportion of excessively large polymer particles should therefore likewise be small.

Excessively large polymer particles are therefore typically removed and recycled into the grinding of the dried polymer gel.

To further improve the properties, the polymer particles may optionally be surface post-crosslinked. Suitable surface post-crosslinkers are compounds which comprise groups which can form covalent bonds with at least two carboxylate groups of the polymer particles. Suitable compounds are, for example, polyfunctional amines, polyfunctional amido amines, polyfunctional epoxides, as described in EP 0 083 022 A2, EP 0 543 303 A1 and EP 0 937 736 A2, di- or polyfunctional alcohols, as described in DE 33 14 019 A1, DE 35 23 617 A1 and EP 0 450 922 A2, or β-hydroxyalkylamides, as described in DE 102 04 938 A1 and U.S. Pat. No. 6,239,230.

Additionally described as suitable surface post-crosslinkers are cyclic carbonates in DE 40 20 780 C1, 2-oxazolidone and its derivatives, such as 2-hydroxyethyl-2-oxazolidone in DE 198 07 502 A1, bis- and poly-2-oxazolidinones in DE 198 07 992 C1, 2-oxotetrahydro-1,3-oxazine and its derivatives in DE 198 54 573 A1, N-acyl-2-oxazolidones in DE 198 54 574 A1, cyclic ureas in DE 102 04 937 A1, bicyclic amide acetals in DE 103 34 584 A1, oxetanes and cyclic ureas in EP 1 199 327 A2 and morpholine-2,3-dione and its derivatives in WO 2003/31482 A1.

Preferred surface post-crosslinkers are ethylene carbonate, ethylene glycol diglycidyl ether, reaction products of polyamides with epichlorohydrin and mixtures of propylene glycol and 1,4-butanediol.

Very particularly preferred surface post-crosslinkers are 2-hydroxyethyloxazolidin-2-one, oxazolidin-2-one and 1,3-propanediol.

In addition, it is also possible to use surface post-crosslinkers which comprise additional polymerisable ethylenically unsaturated groups, as described in DE 37 13 601 A1.

The amount of surface post-crosslinker is preferably 0.001 to 2% by weight, more preferably 0.02 to 1% by weight and most preferably 0.05 to 0.2% by weight, based in each case on the polymer particles.

In a preferred embodiment of the present invention, polyvalent cations are applied to the particle surface in addition to the surface post-crosslinkers before, during or after the surface post-crosslinking.

The polyvalent cations usable in the process according to the invention are, for example, divalent cations such as the cations of zinc, magnesium, calcium, iron and strontium, trivalent cations such as the cations of aluminium, iron, chromium, rare earths and manganese, tetravalent cations such as the cations of titanium and zirconium. Possible counter ions are chloride, bromide, sulphate, hydrogen sulphate, carbonate, hydrogen carbonate, nitrate, phosphate, hydrogen phosphate, dihydrogen phosphate and carboxylate, such as acetate and lactate. Aluminium sulphate is preferred. Apart from metal salts, it is also possible to use polyamines as polyvalent cations.

The amount of polyvalent cation used is, for example, 0.001 to 1.5% by weight, preferably 0.005 to 1% by weight and more preferably 0.02 to 0.8% by weight, based in each case on the polymer particles.

The surface post-crosslinking is typically performed in such a way that a solution of the surface post-crosslinker is sprayed onto the dried polymer particles. After the spraying, the polymer particles coated with the surface post-crosslinker are dried thermally, and the surface post-crosslinking reaction can take place either before or during the drying.

The spraying of a solution of the surface post-crosslinker is preferably performed in mixers with moving mixing tools, such as screw mixers, disk mixers and paddle mixers. Particular preference is given to horizontal mixers such as paddle mixers, very particular preference to vertical mixers. The distinction between horizontal mixers and vertical mixers is made by the position of the mixing shaft, i.e. horizontal mixers have a horizontally mounted mixing shaft and vertical mixers a vertically mounted mixing shaft. Suitable mixers are, for example, horizontal Pflugschar® mixers (Gebr. Lodige Maschinenbau GmbH; Paderborn; Germany), Vrieco-Nauta continuous mixers (Hosokawa Micron BV; Doetinchem; the Netherlands), Processall Mixmill mixers (Processall Incorporated; Cincinnati; US) and Schugi Flexomix® (Hosokawa Micron BV; Doetinchem; the Netherlands). However, it is also possible to spray on the surface post-crosslinker solution in a fluidised bed.

The surface post-crosslinkers are typically used in the form of an aqueous solution. The penetration depth of the surface post-crosslinker into the polymer particles can be adjusted via the content of non-aqueous solvent and total amount of solvent.

When exclusively water is used as the solvent, a surfactant is advantageously added. This improves the wetting behaviour and reduces the tendency to form lumps. However, preference is given to using solvent mixtures, for example isopropanol/water, 1,3-propanediol/water and propylene glycol/water, where the mixing ratio in terms of mass is preferably from 20:80 to 40:60.

The thermal drying (sometimes referred to as “heat-treating” to distinguish this process step from the step of drying the product of the polymerisation, where typically far more water has to be removed; and although embodiments are contemplated in the context of this invention where heat-treating as part of the post-crosslinking step and heat-treating following the step of coating with an elastic film-forming polymer are jointly conducted as one heating step, these heattreatments serve the different functions of completing the post-crosslinking reaction and completing the coating step and must not be confused) is preferably carried out in contact driers, more preferably paddle driers, most preferably disk driers. Suitable driers are, for example, Hosokawa Bepex® horizontal paddle driers (Hosokawa Micron GmbH; Leingarten; Germany), Hosokawa Bepex® disk driers (Hosokawa Micron GmbH; Leingarten; Germany) and Nara paddle driers (NARA Machinery Europe; Frechen; Germany). Moreover, it is also possible to use fluidised bed driers.

The drying can be performed in the mixer itself, by heating the jacket or blowing in warm air. Equally suitable is a downstream drier, for example a shelf drier, a rotary tube oven or a heatable screw. It is particularly advantageous to mix and dry in a fluidised bed drier.

As described above for drying the foam, the optimum temperature and the optimum residence time in the dryer obviously depend on the type of dryer used, the foam thickness and whether there is any forced gas stream in the dryer, and a lower temperature can be offset by a longer residence time and vice versa. In the post-crosslinking step, however, it is important to select drying conditions that, besides being suitable to remove water or other solvents, also allow the post-crosslinking reaction to happen. Less reactive post-crosslinkers need higher temperatures and/or longer residence time than more reactive post-crosslinkers. These parameters can easily be optimised by routine experiments. In typical fluidised bed or contact dryers, the drying temperatures are typically generally in the range of 100 to 250° C., preferably 120 to 220° C., more preferably 150 to 210° C., most preferably 160 to 200° C. The preferred average residence time at this temperature in the dryer is preferably at least 10 minutes, more preferably at least 20 minutes, most preferably at least 30 minutes, and typically at most 120 minutes, more preferably at most 100 minutes, most preferably at most 70 minutes.

Subsequently, the surface post-crosslinked polymer particles can be classified again, excessively small and/or excessively large polymer particles being removed and recycled into the process.

In a preferred embodiment, the surface post-crosslinking is performed as early as the stage of the polymeric foam, in which case the amounts and temperatures specified for the polymer particles apply correspondingly to the polymeric foam.

Following drying, grinding and classifying and before or after, preferably after any surface post-crosslinking, the water-absorbing polymer particles are spray-coated with an elastic film-forming polymer in a fluidised bed reactor in the range from 0° C. to 50° C. and thereafter heat-treated at a temperature above 50° C.

The elastic film-forming polymers to be used according to the present invention for coating are film forming and have elastomeric properties. Polymers having film-forming and also elastic properties are generally suitable, such as copolyesters, copolyamides, silicones, styreneisoprene block copolymers, styrene-butadiene block copolymers and preferably polyurethanes.

“Film forming” means that the respective polymer can readily be made into a layer or coating upon evaporation of the solvent in which it is dissolved or dispersed. The polymer may for example be thermoplastic and/or crosslinked. Elastomeric means the material will exhibit stress induced deformation that is partially or completely reversed upon removal of the stress.

In one embodiment, the polymer has a tensile stress at break in the wet state of at least 1 MPa, or even at least 3 MPa and more preferably at least 5 MPa, or even at least 8 MPa. Most preferred materials have tensile stress at break of at least 10 MPa, preferably at least 40 MPa. This can be determined by the test method, described below.

In one embodiment, particularly preferred polymers herein are materials that have a wet secant elastic modulus at 400% elongation (SMwet 400%) of at least 0.25 MPa, preferably at least about 0.50 MPa, more preferably at least about 0.75 or even at least 2.0 MPa, and most preferably of at least about 3.0 MPa as determined by the test method below.

In one embodiment, preferred polymers herein have a ratio of [wet secant elastic modulus at 400% elongation (SMwet 400%)] to [dry secant elastic modulus at 400% elongation (SMdry 400%)] of 10 or less, preferably of 1.4 or less, more preferably 1.2 or less or even more preferably 1.0 or less, and it may be preferred that this ratio is at least 0.1, preferably at least 0.6, or even at least 0.7.

In one embodiment, the film-forming polymer is present in the form of a coating that has a shell tension, which is defined as the (Theoretical equivalent shell calliper)×(Average wet secant elastic modulus at 400% elongation) of about 5 to 200 N/m, or preferably of 10 to 170 N/m, or more preferably 20 to 130 N/m, and even more preferably 40 to 110 N/m.

In one embodiment of the invention where the water-absorbing polymer particles herein have been surface post-crosslinked (either prior to application of the shell described herein, or at the same time as applying said shell), it may even be more preferred that the shell tension of the water-absorbing material is in the range from 15 N/m to 60 N/m, or even more preferably from 20 N/m to 60 N/m, or preferably from 40 to 60 N/m.

In yet another embodiment wherein the water absorbing polymeric particles are not surface post-crosslinked, it is even more preferred that the shell tension of the water-absorbing material is in the range from about 60 to 110 N/m.

In one embodiment, the film-forming polymer is present in the form of a coating on the surface of the water absorbing material, that has a shell impact parameter, which is defined as the (Average wet secant elastic modulus at 400% elongation)*(Relative Weight of the shell polymer compared to the total weight of the coated polymer) of about 0.03 MPa to 0.6 MPa, preferably 0.07 MPa to 0.45 MPa, more preferably about 0.1 to 0.35 MPa. The “relative weight of the shell polymer compared to the total weight of the coated polymer” is a fraction typically from 0.0 to 1.0.

The water absorbing polymeric particles resulting from the inventive process show an unusual beneficial combination of permeability and swelling speed.

In one embodiment, preference is given to film-forming polymers, especially polyurethanes which, in contrast to the water-absorbing polymeric particles, swell only little if at all on contact with aqueous fluids. This means in practice that the film-forming polymers have preferably a water-swelling capacity of less than 1 g/g, or even less than 0.5 g/g, or even less than 0.2 g/g or even less than 0.1 g/g, as may be determined by the CRC test method, as set out below.

In another embodiment preference is given to film-forming polymers especially polyurethanes which, in contrast to the water-absorbing polymeric particles, swell only moderately on contact with aqueous fluids. This means in practice that the film-forming polymers have preferably a water-swelling capacity of at least 1 g/g, or more than 2 g/g, or even more than 3 g/g, or preferably 4 to 10 g/g, but less than 30 g/g, preferably less than 20 g/g, most preferably less than 12 g/g, as may be determined by the CRC test method, as set out below.

The film forming polymer is typically such that the resulting coating on the water-swellable polymers herein is not water-soluble and, preferably not water-dispersible.

In one embodiment, the polymer is preferably such that the resulting coating on the water-swellable polymers herein is water-permeable, but not water-soluble and, preferably not water-dispersible. Preferably, the polymer especially the polyurethane (tested in the form of a film of a specific calliper, as described herein) is at least moderately water-permeable (breathable) with a moisture vapour transmission rate (MVTR) of more than 200 g/m²/day, preferably breathable with a MVTR of 800 g/m²/day or more preferably 1200 to (inclusive) 1400 g/m²/day, even more preferably breathable with a MVTR of at least 1500 g/m²/day, up to 2100 g/m²/day (inclusive), and most preferably the coating agent or material is highly breathable with a MVTR of 2100 g/m²/day or more.

In order to impart desirable properties to the elastic polymer, additionally fillers such as particulates, oils, solvents, plasticisers, surfactants and/or dispersants may be optionally incorporated.

The mechanical properties as described above are believed to be characteristic in certain embodiments for a suitable film-forming polymer for coating. The polymer may be hydrophobic or hydrophilic. If fast wetting is desired, it is preferable that the polymer is also hydrophilic.

The film-forming polymer can for example be applied from a solution or an aqueous solution or in another embodiment can be applied as dispersion or in a preferred embodiment as aqueous dispersion. The solution can be prepared using any suitable organic solvent for example acetone, isopropanol, tetrahydrofuran, methyl ethyl ketone, dimethyl sulphoxide, dimethyl formamide, chloroform, ethanol, methanol and mixtures thereof.

Polymers can also be blended prior to coating by blending their respective solutions or their respective dispersions. In particular, polymers that do not fulfil the elastic criteria or permeability criteria by themselves can be blended with polymers that do fulfil these criteria and yield a blend that is suitable for coating in the present invention.

Suitable elastomeric polymers which are applicable from solution are for example Vector® 4211 (Dexco Polymers, Texas, USA), Vector 4111, Septon 2063 (Septon Company of America, A Kuraray Group Company), Septon 2007, Estane® 58245 (Noveon, Cleveland, USA), Estane 4988, Estane 4986, Estane® X-1007, Estane T5410, Irogran PS370-201 (Huntsman Polyurethanes), Irogran VP 654/5, Pellethane 2103-70A (Dow Chemical Company), Elastollan® LP 9109 (Elastogran).

In a preferred embodiment the polymer is in the form of an aqueous dispersion and in a more preferred embodiment the polymer is an aqueous dispersion of polyurethane.

The synthesis of polyurethanes and the preparation of polyurethane dispersions are well described for example in Ullmann's Encyclopedia of Industrial Chemistry, Sixth Edition, 2000 Electronic Release.

The polyurethane is preferably hydrophilic and in particular surface hydrophilic. The surface hydrophilicity may be determined by methods known to those skilled in the art. In a preferred execution, the hydrophilic polyurethanes are materials that are wetted by the liquid that is to be absorbed (0.9% saline; urine). They may be characterised by a contact angle that is less than 90 degrees. Contact angles can for example be measured with the Video-based contact angle measurement device, Krüss G10-G1041, available from Kruess, Germany or by other methods known in the art.

In a preferred embodiment, the hydrophilic properties are achieved as a result of the polyurethane comprising hydrophilic polymer blocks, for example polyether groups having a fraction of groups derived from ethylene glycol (CH₂CH₂O) or from 1,4-butane diol (CH₂CH₂CH₂CH₂O) or from propylene glycol (CH₂CH₂CH₂O), or mixtures thereof. Polyether polyurethanes are therefore preferred film-forming polymers. The hydrophilic blocks can be constructed in the manner of comb polymers where parts of the side chains or all side chains are hydrophilic polymeric blocks. But the hydrophilic blocks can also be constituents of the main chain (i.e., of the polymer's backbone). In a preferred embodiment, polyurethanes are used where at least the predominant fraction of the hydrophilic polymeric blocks is present in the form of side chains. The side chains can in turn be block copolymers such as poly(ethylene glycol)-co-poly(propylene glycol).

It is further possible to obtain hydrophilic properties for the polyurethanes through an elevated fraction of ionic groups, preferably carboxylate, sulphonate, phosphonate or ammonium groups. The ammonium groups may be protonated or alkylated tertiary or quaternary groups. Carboxylates, sulphonates, and phosphates may be present as alkali-metal or ammonium salts. Suitable ionic groups and their respective precursors are for example described in “Ullmanns Encyclopädie der technischen Chemie”, 4th Edition, Volume 19, p. 311-313 and are furthermore described in DE-A 1 495 745 and WO 03/050156.

The hydrophilicity of the preferred polyurethanes facilitates the penetration and dissolution of water into the water-absorbing polymeric particles which are enveloped by the film-forming polymer. The present invention's coatings with these preferred polyurethanes are notable for the fact that the mechanical properties are not excessively impaired even in the moist state, despite the hydrophilicity.

Preferred film forming polymers have two or more glass transition temperatures Tg (determined by DSC). Ideally, the polymers used exhibit the phenomenon of phase separation, i.e., they contain two or more different blocks of low and high Tg side by side in the polymer (Thermoplastic Elastomers: A Comprehensive Review, eds. Legge, N. R., Holden, G., Schroeder, H. E., 1987, chapter 2). However, the measurement of Tg may in practice be very difficult in cases when several glass transition temperatures are close together or for other experimental reasons. Even in cases when the glass transition temperatures cannot be determined clearly by experiment the polymer may still be suitable in the scope of the present invention.

Especially preferred phase-separating polymers, and especially polyurethanes, herein comprise one or more phase-separating block copolymers, having a weight average molecular weight Mw of at least 5 kg/mol, preferably at least 10 kg/mol and higher.

In one embodiment such a block copolymer has at least a first polymerised homopolymer segment (block) and a second polymerised homopolymer segment (block), polymerised with one another, whereby preferably the first (soft) segment has a Tg1 of less than 25° C. or even less than 20° C., or even less than 0° C., and the second (hard) segment has a Tg2 of at least 50° C., or of 55° C. or more, preferably 60° C. or more or even 70° C. or more.

In another embodiment, especially with polyurethanes, such a block copolymer has at least a first polymerised heteropolymer segment (block) and a second polymerised heteropolymer segment (block), polymerised with one another, whereby preferably the first (soft) segment has a Tg1 of less than 25° C. or even less than 20° C., or even less than 0° C., and the second (hard) segment has a Tg2 of at least 50° C., or of 55° C. or more, preferably 60° C. or more or even 70° C. or more.

In one embodiment the total weight average molecular weight of the hard second segments (with a Tg of at least 50° C.) is preferably at least 28 kg/mol, or even at least 45 kg/mol.

The preferred weight average molecular weight of a first (soft) segment (with a Tg of less than 25° C.) is at least 500 g/mol, preferably at least 1000 g/mol or even at least 2000 g/mol, but preferably less than 8000 g/mol, preferably less than 5000 g/mol.

However, the total of the first (soft) segments is typically 20% to 95% by weight of the total block copolymer, or even from 20% to 85% or more preferably from 30% to 75% or even from 40% to 70% by weight. Furthermore, when the total weight level of soft segments is more than 70%, it is even more preferred that an individual soft segment has a weight average molecular weight of less than 5000 g/mol.

It is well understood by those skilled in the art that “polyurethanes” is a generic term used to describe polymers that are obtained by reacting di- or polyisocyanates with at least one di- or polyfunctional “active hydrogen-containing” compound. “Active hydrogen containing” means that the di- or polyfunctional compound has at least 2 functional groups which are reactive toward isocyanate groups (also referred to as reactive groups), e.g. hydroxyl groups, primary and secondary amino groups and mercapto (SH) groups.

It also is well understood by those skilled in the art that polyurethanes also include allophanate, biuret, carbodiimide, oxazolidinyl, isocyanurate, uretdione, and other linkages in addition to urethane and urea linkages.

In one embodiment the block copolymers useful herein are preferably polyether urethanes and polyester urethanes. Polyether urethanes comprising polyalkylene glycol units, especially polyethylene glycol units or poly(tetramethylene glycol) units are especially preferred.

As used herein, the term “alkylene glycol” includes both alkylene glycols and substituted alkylene glycols having 2 to 10 carbon atoms, such as ethylene glycol, 1,3 propylene glycol, 1,2-propylene glycol, 1,2-butylene glycol, 1,3-butylene glycol, 1,4 butylene glycol, styrene glycol and the like.

The polyurethanes used according to the present invention are generally obtained by reaction of polyisocyanates with active hydrogen-containing compounds having two or more reactive groups. These include high molecular weight compounds having a molecular weight in the range of preferably 300 to 100 000 g/mol especially from 500 to 30 000 g/mol, low molecular weight compounds and compounds having polyether groups, especially polyethylene oxide groups or polytetrahydro furan groups and a molecular weight in the range from 200 to 20 000 g/mol, the polyether groups in turn having no reactive groups.

These compounds can also be used as mixtures.

Suitable polyisocyanates have an average of about two or more isocyanate groups, preferably an average of about two to about four isocyanate groups and include aliphatic, cycloaliphatic, araliphatic, and aromatic polyisocyanates, used alone or in mixtures of two or more. Diisocyanates are more preferred. Aliphatic and cycloaliphatic polyisocyanates, especially diisocyanates, are especially preferred.

Specific examples of suitable aliphatic diisocyanates include alpha, omega-alkylene diisocyanates having from 5 to 20 carbon atoms, such as hexamethylene-1,6-diisocyanate, 1,12-dodecane diisocyanate, 2,2,4-trimethylhexamethylene diisocyanate, 2,4,4-trimethyl-hexamethylene diisocyanate, 2-methyl-1,5-pentamethylene diisocyanate, and the like. Polyisocyanates having fewer than 5 carbon atoms can be used but are less preferred because of their high volatility and toxicity. Preferred aliphatic polyisocyanates include hexamethylene-1,6-diisocyanate, 2,2,4-trimethyl-hexamethylene diisocyanate, and 2,4,4-trimethyl-hexamethylene diisocyanate.

Specific examples of suitable cycloaliphatic diisocyanates include dicyclohexyl methane diisocyanate, (commercially available as Desmodur® W from Bayer Corporation), isophorone diisocyanate, 1,4-cyclohexane diisocyanate, 1,3-bis(isocyanatomethyl) cyclohexane, and the like. Preferred cycloaliphatic diisocyanates include dicyclohexyl methane diisocyanate and isophorone diisocyanate.

Specific examples of suitable araliphatic diisocyanates include m-tetramethyl xylylene diisocyanate, p-tetramethyl xylylene diisocyanate, 1,4-xylylene diisocyanate, 1,3-xylylene diisocyanate, and the like. A preferred araliphatic diisocyanate is tetramethyl xylylene diisocyanate.

Examples of suitable aromatic diisocyanates include 4,4′-diphenylmethane diisocyanate, toluene diisocyanate, their isomers, naphthalene diisocyanate, and the like. A preferred aromatic diisocyanate is toluene diisocyanate and 4,4′-diphenylmethane diisocyanate.

Examples of high molecular weight compounds having 2 or more reactive groups are such as polyester polyols and polyether polyols, as well as polyhydroxy polyester amides, hydroxyl-containing polycaprolactones, hydroxyl-containing acrylic copolymers, hydroxyl-containing epoxides, polyhydroxy polycarbonates, polyhydroxy polyacetals, polyhydroxy polythioethers, polysiloxane polyols, ethoxylated polysiloxane polyols, polybutadiene polyols and hydrogenated polybutadiene polyols, polyacrylate polyols, halogenated polyesters and polyethers, and the like, and mixtures thereof. The polyester polyols, polyether polyols, polycarbonate polyols, polysiloxane polyols, and ethoxylated polysiloxane polyols are preferred. Particular preference is given to polyester polyols, polycarbonate polyols and polyalkylene ether polyols. The number of functional groups in the aforementioned high molecular weight compounds is preferably on average in the range from 1.8 to 3 and especially in the range from 2 to 2.2 functional groups per molecule.

The polyester polyols typically are esterification products prepared by the reaction of organic polycarboxylic acids or their anhydrides with a stoichiometric excess of a diol.

The diols used in making the polyester polyols include alkylene glycols, e.g., ethylene glycol, 1,2- and 1,3-propylene glycols, 1,2-, 1,3-, 1,4-, and 2,3-butane diols, hexane diols, neopentyl glycol, 1,6-hexanediol, 1,8-octanediol, and other glycols such as bisphenol-A, cyclohexanediol, cyclohexane dimethanol (1,4-bis-hydroxymethylcyco-hexane), 2-methyl-1,3-propanediol, 2,2,4-trimethyl-1,3-pentanediol, diethylene glycol, triethylene glycol, tetraethylene glycol, polyethylene glycol, dipropylene glycol, polypropylene glycol, dibutylene glycol, polybutylene glycol, dimerate diol, hydroxylated bisphenols, polyether glycols, halogenated diols, and the like, and mixtures thereof. Preferred diols include ethylene glycol, diethylene glycol, butane diol, hexane diol, and neopentyl glycol. Alternatively or in addition, the equivalent mercapto compounds may also be used.

Suitable carboxylic acids used in making the polyester polyols include dicarboxylic acids and tricarboxylic acids and anhydrides, e.g., maleic acid, maleic anhydride, succinic acid, glutaric acid, glutaric anhydride, adipic acid, suberic acid, pimelic acid, azelaic acid, sebacic acid, chlorendic acid, 1,2,4-butane-tricarboxylic acid, phthalic acid, the isomers of phthalic acid, phthalic anhydride, fumaric acid, dimeric fatty acids such as oleic acid, and the like, and mixtures thereof. Preferred polycarboxylic acids used in making the polyester polyols include aliphatic or aromatic dibasic acids.

Examples of suitable polyester polyols include poly(glycol adipate)s, poly(ethylene terephthalate)polyols, polycaprolactone polyols, orthophthalic polyols, sulphonated and phosphonated polyols, and the like, and mixtures thereof.

The preferred polyester polyol is a diol. Preferred polyester diols include poly(butanediol adipate); hexanediol adipic acid and isophthalic acid polyesters such as hexaneadipate isophthalate polyester; hexanediol neopentyl glycol adipic acid polyester diols, e.g., Piothane 67-3000 HNA (Panolam Industries) and Piothane 67-1000 HNA, as well as propylene glycol maleic anhydride adipic acid polyester diols, e.g., Piothane SO-1000 PMA, and hexane diol neopentyl glycol fumaric acid polyester diols, e.g., Piothane 67-SO0 HNF. Other preferred Polyester diols include Rucoflex® S101.5-3.5, S1040-3.5, and S-1040-110 (Bayer Corporation).

Polyether polyols are obtained in known manner by the reaction of a starting compound that contain reactive hydrogen atoms, such as water or the diols set forth for preparing the polyester polyols, and alkylene glycols or cyclic ethers, such as ethylene glycol, propylene glycol, butylene glycol, styrene glycol, ethylene oxide, propylene oxide, 1,2-butylene oxide, 2,3-butylene oxide, oxetane, tetrahydrofuran, epichlorohydrin, and the like, and mixtures thereof. Preferred polyethers include poly(ethylene glycol), poly(propylene glycol), polytetrahydro furan, and co[poly(ethylene glycol)-poly(propylene glycol)]. Polyethylene glycol and Polypropylene glycol can be used as such or as physical blends. In case that propylene oxide and ethylene oxide are copolymerised, these polypropylene-co-polyethylene polymers can be used as random polymers or block-copolymers.

In one embodiment the polyether polyol is a constituent of the main polymer chain.

In another embodiment the polyetherol is a terminal group of the main polymer chain.

In yet another embodiment the polyether polyol is a constituent of a side chain which is comb-like attached to the main chain. An example of such a monomer is Tegomer D-3403 (Degussa). Polycarbonates include those obtained from the reaction of diols such 1,3-propanediol, 1,4-butanediol, 1,6-hexanediol, diethylene glycol, triethylene glycol, tetraethylene glycol, and the like, and mixtures thereof with dialkyl carbonates such as diethyl carbonate, diaryl carbonates such as diphenyl carbonate or phosgene.

Examples of low molecular weight compounds having two reactive functional groups are the diols such as alkylene glycols and other diols mentioned above in connection with the preparation of polyester polyols. They also include diamines such as diamines and polyamines which are among the preferred compounds useful in preparing the aforesaid polyester amides and polyamides. Suitable diamines and polyamines include 1,2-diaminoethane, 1,6-diaminohexane, 2-methyl-1,5-pentanediamine, 2,2,4-trimethyl-1,6-hexanediamine, 1,12-diaminododecane, 2-aminoethanol, 2-[(2-aminoethyl)amino]-ethanol, piperazine, 2,5-dimethylpiperazine, 1-amino-3-aminomethyl-3,5,5-trimethylcyclohexane (isophorone diamine or IPDA), bis-(4-aminocyclohexyl)-methane, bis-(4-amino-3-methyl-cyclohexyl)-methane, 1,4-diaminocyclohexane, 1,2-propylenediamine, hydrazine, urea, amino acid hydrazides, hydrazides of semicarbazido carboxylic acids, bis-hydrazides and bis-semicarbazides, diethylene triamine, triethylene tetramine, tetraethylene pentamine, pentaethylene hexamine, N,N,N-tris-(2-aminoethyl)amine, N-(2-piperazinoethyl)-ethylene diamine, N,N′-bis-(2-aminoethyl)-piperazine, N,N,N′-tris-(2-aminoethyl)ethylene diamine, N-[N-(2-aminoethyl)-2-aminoethyl]-N′-(2-aminoethyl)-piperazine, N-(2-aminoethyl)-N′-(2-piperazinoethyl)-ethylene diamine, N,N-bis-(2-aminoethyl)-N-(2-piperazinoethyl)amine, N,N-bis-(2-piperazinoethyl)amine, polyethylene imines, iminobispropyl amine, guanidine, melamine, N-(2-aminoethyl)-1,3-propane diamine, 3,3′-diaminobenzidine, 2,4,6-triaminopyrimidine, polyoxypropylene amines, tetrapropylene pentamine, tripropylene tetramine, N,N-bis-(6-aminohexyl)amine, N,N′-bis-(3-aminopropyl)ethylene diamine, and 2,4-bis-(4′-aminobenzyl)-aniline, and the like, and mixtures thereof. Preferred diamines and polyamines include 1-amino-3-aminomethyl-3,5,5-trimethyl-cyclohexane (isophorone diamine or IPDA), bis-(4-am inocyclohexyl)-methane, bis-(4-amino-3-methylcyclohexyl)methane, ethylene diamine, diethylene triamine, triethylene tetramine, tetraethylene pentamine, and pentaethylene hexamine, and the like, and mixtures thereof. Other suitable diamines and polyamines for example include Jeffamine® D-2000 and D-4000, which are amine-terminated polypropylene glycols differing only by molecular weight, and Jeffamine XTJ-502, T 403, T 5000, and T 3000 which are amine terminated polyethylene glycols, amine terminated copolypropylene-polyethylene glycols, and triamines based on propoxylated glycerol or trimethylolpropane and which are available from Huntsman Chemical Company.

The poly(alkylene glycol) may be part of the polymer main chain or be attached to the main chain in comb-like shape as a side chain.

In a preferred embodiment, the polyurethane comprises poly(alkylene glycol) side chains sufficient in amount to comprise about 10 wt. % to 90 wt. %, preferably about 12 wt. % to about 80 wt. %, preferably about 15 wt. % to about 60 wt. %, and more preferably about 20 wt. % to about 50 wt. %, of poly(alkylene glycol) units in the final polyurethane on a dry weight basis. At least about 50 wt. %, preferably at least about 70 wt. %, and more preferably at least about 90 wt. % of the poly(alkylene glycol) side-chain units comprise poly(ethylene glycol), and the remainder of the side-chain poly-(alkylene glycol) units can comprise alkylene glycol and substituted alkylene glycol units having from 3 to about 10 carbon atoms. The term “final polyurethane” means the polyurethane used for coating the water-absorbing polymeric particles.

Preferably the amount of the side-chain units is (i) at least about 30 wt. % when the molecular weight of the side-chain units is less than about 600 g/mol, (ii) at least about 15 wt. % when the molecular weight of the side-chain units is from about 600 to about 1000 g/mol, and (iii) at least about 12 wt. % when the molecular weight of said side-chain units is more than about 1000 g/mol. Mixtures of active hydrogen-containing compounds having such poly(alkylene glycol) side chains can be used with active hydrogen-containing compounds not having such side chains.

These side chains can be incorporated in the polyurethane by replacing a part or all of the aforementioned high molecular diols or low molecular compounds by compounds having at least two reactive functional groups and a polyether group, preferably a polyalkylene ether group, more preferably a polyethylene glycol group that has no reactive group.

For example, active hydrogen-containing compounds having a polyether group, in particular a poly(alkylene glycol) group, include diols having poly(ethylene glycol) groups such as those described in U.S. Pat. No. 3,905,929 that is incorporated herein by reference in its entirety. Further, U.S. Pat. No. 5,700,867 that is incorporated herein by reference in its entirety teaches methods for incorporation of poly(ethylene glycol) side chains at col. 4, line 3.5 to col. 5, line 4.5. A preferred active hydrogen-containing compound having poly(ethylene glycol) side chains is trimethylol propane mono (polyethylene oxide methyl ether), available as Tegomer D-3403 from Degussa-Goldschmidt.

Preferably, the polyurethanes to be used in the present invention also have reacted therein at least one active hydrogen-containing compound not having said side chains and typically ranging widely in molecular weight from about 50 to about 10000 g/mol, preferably about 200 to about 6000 g/mol, and more preferably about 300 to about 3000 g/mol. Suitable active hydrogen-containing compounds not having said side chains include any of the amines and polyols described herein above.

According to one preferred embodiment of the invention, the active hydrogen compounds are chosen to provide less than about 25 wt. %, more preferably less than about 15 wt. % and most preferably less than about 5 wt. % poly(ethylene glycol) units in the backbone (main chain) based upon the dry weight of final polyurethane, since such main-chain poly(ethylene glycol) units tend to cause swelling of polyurethane particles in the waterborne polyurethane dispersion and also contribute to lower in use tensile strength of articles made from the polyurethane dispersion.

The preparation of polyurethanes having polyether side chains is known to one skilled in the art and is extensively described for example in US 2003/0195293, which is hereby expressly incorporated herein by reference.

The present invention accordingly also provides a water-absorbing material comprising water-absorbing polymeric particles coated with elastic film-forming polyurethane, wherein the polyurethane comprises not only side chains having polyethylene oxide units but also polyethylene oxide units in the main chain.

Advantageous polyurethanes within the realm of this invention are obtained by first preparing prepolymers having isocyanate end groups, which are subsequently linked together in a chainextending step. The linking together can be through water or through reaction with a compound having at least one crosslinkable functional group.

The prepolymer is obtained by reacting one of the above-described isocyanate compounds with an active hydrogen compound. Preferably the prepolymer is prepared from the abovementioned polyisocyanates, at least one compound c) and optionally at least one further active hydrogen compound selected from the compounds a) and b).

In one embodiment the ratio of isocyanate to active hydrogen in the compounds forming the prepolymer typically ranges from about 1.3/1 to about 2.5/1, preferably from about 1.5/1 to about 2.1/1, and more preferably from about 1.7/1 to about 2/1.

The polyurethane may additionally contain functional groups which can undergo further crosslinking reactions and which can optionally render them self-crosslinkable.

Compounds having at least one additional crosslinkable functional group include those having carboxylic, carbonyl, amine, hydroxyl, and hydrazide groups, and the like, and mixtures of such groups. The typical amount of such optional compound is up to about 1 milliequivalent, preferably from about 0.05 to about 0.5 milliequivalents, and more preferably from about 0.1 to about 0.3 milliequivalents per gram of final polyurethane on a dry weight basis.

The preferred monomers for incorporation into the isocyanate-terminated prepolymer are hydroxy-carboxylic acids having the general formula (HO)xQ(COOH)y wherein Q is a straight or branched hydrocarbon radical having 1 to 12 carbon atoms, and x and y are 1 to 3. Examples of such hydroxy-carboxylic acids include citric acid, dimethylolpropanoic acid (DMPA), dimethylol butanoic acid (DMBA), glycolic acid, lactic acid, malic acid, dihydroxymalic acid, tartaric acid, hydroxypivalic acid, and the like, and mixtures thereof. Dihydroxy-carboxylic acids are more preferred with dimethylolpropanoic acid (DMPA) being most preferred.

Other suitable compounds providing crosslinkability include thioglycolic acid, 2,6-dihydroxybenzoic acid, and the like, and mixtures thereof.

Optional neutralisation of the prepolymer having pendant carboxyl groups will convert the carboxyl groups to carboxylate anions, thus enhancing water-dispersibility. Suitable neutralizing agents include tertiary amines, metal hydroxides, ammonia, and other agents well known to those skilled in the art.

As a chain extender, at least one of water, an inorganic or organic polyamine having an average of about 2 or more primary and/or secondary amine groups, polyalcohols, ureas, or combinetions thereof is suitable for use in the present invention. Suitable organic amines for use as a chain extender include diethylene triamine (DETA), ethylene diamine (EDA), metaxylylenediamine (MXDA), aminoethyl ethanolamine (AEEA), 2-methyl pentane diamine, and the like, and mixtures thereof. Also suitable for practice in the present invention are propylene diamine, butylene diamine, hexamethylene diamine, cyclohexylene diamine, phenylene diamine, tolylene diamine, 3,3-dichlorobenzidene, 4,4′-methylene-bis-(2-chloroaniline), 3,3-dichloro-4,4-diamino diphenylmethane, sulphonated primary and/or secondary amines, and the like, and mixtures thereof. Suitable inorganic and organic amines include hydrazine, substituted hydrazines, and hydrazine reaction products, and the like, and mixtures thereof. Suitable polyalcohols include those having from 2 to 12 carbon atoms, preferably from 2 to 8 carbon atoms, such as ethylene glycol, diethylene glycol, neopentyl glycol, butanediols, hexanediol, and the like, and mixtures thereof. Suitable ureas include urea and its derivatives, and the like, and mixtures thereof. Hydrazine is preferred and is most preferably used as a solution in water. The amount of chain extender typically ranges from about 0.5 to about 0.95 equivalents based on available isocyanate.

A degree of branching of the polyurethane may be beneficial, but is not required to maintain a high tensile strength and improve resistance to creep (cf. strain relaxation). This degree of branching may be accomplished during the prepolymer step or the extension step. For branching during the extension step, the chain extender DETA is preferred, but other amines having an average of about two or more primary and/or secondary amine groups may also be used. For branching during the prepolymer step, it is preferred that trimethylol propane (TMP) and other polyols having an average of more than two hydroxyl groups be used. The branching monomers can be present in amounts up to about 4 wt. % of the polymer backbone.

Polyurethanes are preferred film-forming polymers. They can be applied to the water-absorbing polymer particles as solution in a solvent or as dispersion. Particularly preferred are aqueous dispersions.

Preferred aqueous polyurethane dispersions are Hauthane HD-4638 (ex Hauthaway), Hydrolar HC 269 (ex Colm, Italy), Impraperm 48180 (ex Bayer Material Science AG, Germany), Lupraprot DPS (ex BASF Germany), Permax 120, Permax 200, and Permax 220 (ex Noveon, Brecksville, Ohio),), Syntegra YM2000 and Syntegra YM2100 (ex Dow, Midland, Mich.) Witcobond G-213, Witcobond G-506, Witcobond G-507, and Witcobond 736 (ex Uniroyal Chemical, Middlebury, Conn.).

Particularly suitable elastic film-forming polyurethanes are extensively described in the literature references herein below and expressly form part of the subject matter of the present disclosure.

Particularly hydrophilic thermoplastic polyurethanes are sold by Noveon, Brecksville, Ohio, under the trade names of Permax 120, Permax 200 and Permax 220 and are described in detail in “Proceedings International Waterborne High Solids Coatings, 32, 299, 2004” and were presented to the public in February 2004 at the “International Waterborne, High-Solids, and Powder Coatings Symposium” in New Orleans, USA. The preparation is described in detail in US 2003/0195293.

Furthermore, the polyurethanes described in U.S. Pat. No. 4,190,566, U.S. Pat. No. 4,092,286, US 2004/0214937 and also WO 03/050156 expressly form part of the subject matter of the present disclosure.

More particularly, the polyurethanes described can be used in mixtures with each other or with other film-forming polymers, fillers, oils, water-soluble polymers or plasticizing agents in order that particularly advantageous properties may be achieved with regard to hydrophilicity, water perviousness and mechanical properties.

It may be preferred that the coating agent herein comprises fillers to reduce tack such as the commercially available resin Estane 58245-047P and Estane X-1007-040P, available from Noveon Inc., 9911 Brecksville Road, Cleveland, OH44 141-3247, USA.

Alternatively such fillers can be added in order to reduce tack to the dispersions or solutions of suitable elastomeric polymers before application. Aerosil is a typical filler, but other inorganic deagglomeration aids as listed below can also be used.

Preferred polyurethanes for use in the coating agent herein are strain hardening and/or strain crystallizing. Strain Hardening is observed during stress-strain measurements, and is evidenced as the rapid increase in stress with increasing strain. It is generally believed that strain hardening is caused by orientation of the polymer chains in the film producing greater resistance to extension in the direction of drawing.

The coating agent is applied such that the resulting coating layer is preferably thin having an average calliper (thickness) of more than 0.1 μm; preferably the coating layer has an average calliper (thickness) from 1 micron (μm) to 100 microns, preferably from 1 micron to 50 microns, more preferably from 1 micron to 20 microns or even from 2 to 20 microns or even from 2 to 10 microns.

In one embodiment the coating is preferably uniform in calliper and/or shape. Preferably, the average calliper is such that the ratio of the smallest to largest calliper is from 1:1 to 1:5, preferably from 1:1 to 1:3, or even 1:1 to 1:2, or even 1:1 to 1:1.5.

In another embodiment the coating may show some defects (i.e. holes) but still the polymer shows very good performance properties according to the present invention. In yet another embodiment of the invention, the coating may form a fibrous net around the water-absorbing particles.

The polymeric film is preferably applied in an amount of 0.1-25 parts by weight of the film-forming polymer (reckoned as solids material) to 100 parts by weight of dry water-absorbing polymeric particles. The amount of film-forming polymer used per 100 parts by weight of waterabsorbing polymeric particles is preferably 0.1-15 parts by weight, especially 0.5-10 parts by weight, more preferably 0.5-7 parts by weight, even more preferably 0.5-5 parts by weight and in particular 0.5-4.5 parts by weight.

Particular preference is given to a water-absorbing material obtained by coating water-absorbing polymeric particles with <5 parts by weight, preferably 0.5-4.5 parts by weight, especially 0.5-4 parts by weight and more preferably 0.5-3 parts by weight of film-forming polymer based on 100 parts by weight of water-absorbing polymeric particles, preferably at temperatures in the range from 0□C to <50° C., preferably from 0 □C to <45 □C, more preferably from 10 □C to <40 □C, and most preferably from 15 □C to <35 □C, and then heat-treating the coated particles at a temperature above 50° C.

The film-forming polymer especially the polyurethane can be applied as a solid material, as a hot-melt, as a dispersion, as an aqueous dispersion, as an aqueous solution or as an organic solution to the particles of the water-absorbing addition polymer. The form in which the film-forming polymer, especially the polyurethane is applied to the water-absorbing polymeric particles is preferably as a solution or more preferably as an aqueous dispersion.

Useful solvents for polyurethanes include solvents which make it possible to establish 1 to not less than 40% by weight concentrations of the polyurethane in the respective solvent or mixture. As examples there may be mentioned alcohols, esters, ethers, ketones, amides, and halogenated hydrocarbons like methyl ethyl ketone, acetone, isopropanol, tetrahydrofuran, dimethyl formamide, chloroform and mixtures thereof. Solvents which are polar, aprotic and boil below 100° C. are particularly advantageous.

Aqueous herein refers to water and also mixtures of water with up to 20% by weight of water-miscible solvents, based on the total amount of solvent. Water-miscible solvents are miscible with water in the desired use amount at 25° C. and 1 bar. They include alcohols such as methanol, ethanol, propanol, isopropanol, ethylene glycol, 1,2-propanediol, 1,3-propanediol, ethylene carbonate, glycerol and methoxy ethanol and water-soluble ethers such as tetrahydrofuran and dioxane.

It is particularly preferable to perform the coating in a fluidised bed reactor. The water-absorbing particles are introduced as generally customary, depending on the type of the reactor, and are generally coated by spraying with the film-forming polymer as a solid material or preferably as a polymeric solution or dispersion. Aqueous dispersions of the film-forming polymer are particularly preferred for this.

The polyurethane solution or dispersion applied by spray-coating is preferably very concentrated. For this, the viscosity of this polyurethane mixture must not be too high, or the polyurethane solution or dispersion can no longer be finely dispersed for spraying. Preference is given to a polyurethane solution or dispersion having a viscosity of <500 mPa s, preferably of <300 mPa s, more preferably of <100 mPa s, even more preferably of <10 mPa s, and most preferably <5 mPa s (typically determined with a rotary viscometer at a shear rate 200 rpm for the polyurethane dispersion, and specifically suitable is a Haake rotary viscometer type RV20, system M5, NV).

In embodiments in which other film-forming polymers are used, it is preferred that these exhibit the same viscosities as the polyurethanes when applied.

The concentration of polyurethane in the polyurethane solution or dispersion is generally in the range from 1% to 60% by weight, preferably in the range from 5% to 40% by weight and especially in the range from 10% to 30% by weight. Higher dilutions are possible, but generally lead to longer coating times. A particular advantage of polyurethane dispersions is their relatively low viscosity even at high concentrations and high molecular weights.

Useful fluidised bed reactors include for example the fluidised or suspended bed coaters familiar in the pharmaceutical industry. Particular preference is given to the Wurster process and the Glatt-Zeller process and these are described for example in “Pharmazeutische Technologie, Georg Thieme Verlag, 2nd edition (1989), pages 412-413” and also in “Arzneiformenlehre, Wissenschaftliche Verlagsbuchhandlung mbH, Stuttgart 1985, pages 130-132”. Particularly suitable batch and continuous fluidised bed processes on a commercial scale are described in Drying Technology, 20(2), 419-447 (2002).

In the Wurster process the absorbent polymeric particles are carried by an upwardly directed stream of carrier gas in a central tube, against the force of gravity, past at least one spray nozzle and are sprayed concurrently with the finely disperse polymeric solution or dispersion. The particles thereafter fall back to the base along the side walls, are collected on the base, and are again carried by the flow of carrier gas through the central tube past the spray nozzle. The spray nozzle typically sprays from the bottom into the fluidised bed. It may also project from the bottom into the fluidised bed.

In the Glatt-Zeller process, the polymeric particles are conveyed by the carrier gas on the outside along the walls in the upward direction and then fall in the middle onto a central nozzle head, which typically comprises at least 3 two-material nozzles which spray to the side. The particles are thus sprayed from the side, fall past the nozzle head to the base and are taken up again there by the carrier gas, so that the cycle can start anew.

The feature common to the two processes is that the particles are repeatedly carried in the form of a fluidised bed past the spray device, whereby a very thin and typically very homogeneous shell can be applied. Furthermore, a carrier gas is used at all times and it has to be fed and moved at a sufficiently high rate to maintain fluidisation of the particles. As a result, liquids are rapidly vaporised in the apparatus, such as for example the solvent (i.e. water) of the dispersion, even at low temperatures, whereby the polymeric particles of the dispersion are precipitated onto the surface of the particles of the absorbent polymer which are to be coated. Useful carrier gases include the inert gases mentioned above and air or dried air or mixtures of any of these gases.

Suitable fluidised bed reactors work according to the principle that the film-forming polymer solution or dispersion is finely atomised and the droplets randomly collide with the water-absorbing polymer particles in a fluidised bed, whereby a substantially homogeneous shell builds up gradually and uniformly after many collisions. The size of the droplets must be inferior to the particle size of the absorbent polymer. Droplet size is determined by the type of nozzle, the spraying conditions i.e. temperature, concentration, viscosity, pressure and typical droplets sizes are in the range 10 μm to 400 μm. A polymer particle size vs. droplet size ratio of at least 10 is typically observed. Small droplets with a narrow size distribution are favourable. The droplets of the atomised polymeric dispersion or solution are introduced either concurrently with the particle flow or from the side into the particle flow, and may also be sprayed from the top onto a fluidised bed. In this sense, other apparatus and equipment modifications which comply with this principle and which are likewise capable of building up fluidised beds are perfectly suitable for producing such effects.

One embodiment, for example, is a cylindrical fluidised bed batch reactor, in which the water-absorbing polymer particles are transported upwards by a carrier-gas stream at the outer walls inside the apparatus and from one or more positions a film-forming polymer spray is applied from the side into this fluidised bed, whereas in the middle zone of the apparatus, in which there is no carrier gas stream at all and where the particles fall down again, a cubic agitator is moving and redistributing the entire fluidised particle bed.

Other embodiments, for example, may be Schugi Flexomix® (Hosokawa Micron), Turbulizer® (Bepex) or Plowshare® (Lodige) mixers which can be used alone or preferably as a battery of plural consecutive units. If such a mixer is used alone, the water-absorbing polymer may have to be fed multiple times through the apparatus to become homogeneously coated. If two or more of such apparatus are set up as consecutive units then one pass may be sufficient.

In another embodiment continuous or batch-type spray-mixers of the Telschig-type are used in which the spray hits free falling particles in-flight, the particles being repeatedly exposed to the spray. Suitable mixers are described in Chemie-Technik, 22 (1993), Nr. 4, p. 98 if.

In a preferred embodiment, a continuous fluidised bed process is used and the spray is operated in top or bottom-mode. In a particularly preferred embodiment the spray is operated bottom-mode and the process is continuous. A suitable apparatus is for example described in U.S. Pat. No. 5,211,985. Suitable apparatus are available also for example from Glatt Maschinen- and Apparatebau AG (Switzerland) as series GF (continuous fluidised bed) and as ProCell spouted bed. The spouted bed technology uses a simple slot instead of a screen bottom to generate the fluidised bed and is particularly suitable for materials which are difficult to fluidise.

In other embodiments it may also be desired to operate the spray top- and bottom-mode, or it may be desired to spray from the side or from a combination of several different spray positions.

The aforementioned nozzles which are customarily used for post-crosslinking may also be used in the process of the present invention. However, two-material nozzles are particularly preferred.

The process of the present invention is preferably performed in Wurster Coaters. Examples for such coaters are PRECISION COATERS available from GEA-Aeromatic Fielder AG (Switzerland) and are accessible at Coating Place Inc. (Wisconsin, USA).

It is advantageous that the fluidised bed gas stream which enters from below is likewise chosen such that the total amount of the water-absorbing polymeric particles is fluidised in the apparatus. The gas velocity for the fluidised bed is above the minimum fluidisation velocity (measurement method described in Kunii and Levenspiel “Fluidisation engineering” 1991) and below the terminal velocity of water-absorbing polymer particles, preferably 10% above the minimum fluidisation velocity. The gas velocity for the Wurster tube is above the terminal velocity of water-absorbing polymer particles, usually below 100 m/s, preferably 10% above the terminal velocity.

The gas stream acts to vaporise the water, or the solvents. In a preferred embodiment, the coating conditions of gas stream and temperature are chosen so that the relative humidity or vapour saturation at the exit of the gas stream is in the range from 10% to 90%, preferably from 10% to 80%, or preferably from 10% to 70% and especially from 30% to 60%, based on the equivalent absolute humidity prevailing in the carrier gas at the same temperature or, if appropriate, the absolute saturation vapour pressure.

The fluidised bed reactor may be built from stainless steel or any other typical material used for such reactors, also the product contacting parts may be stainless steel to accommodate the use of organic solvents and high temperatures.

In a further preferred embodiment, the inner surfaces of the fluidised bed reactor are at least partially coated with a material whose contact angle with water is more than 90° at 25° C. Teflon or polypropylene are examples of such materials. Preferably, all product-contacting parts of the apparatus are coated with this material.

The choice of material for the product-contacting parts of the apparatus, however, also depends on whether these materials exhibit strong adhesion to the polymeric dispersion or solution used for coating or to the polymers to be coated. Preference is given to selecting materials which have no such adhesion either to the polymer to be coated or to the polymer dispersion or solution in order that caking may be avoided.

According to the present invention, coating takes place at a product and/or carrier gas temperature in the range from 0° C. to 50° C., preferably 5-45° C., especially 10-40° C. and most preferably 15-35° C.

In a preferred embodiment, a deagglomeration aid is added before the heat-treating step to the particles to be coated or preferably which have already been coated. A deagglomeration aid would be known by those skilled in the art to be for example a finely divided water-insoluble salt selected from organic and inorganic salts and mixtures thereof, and also waxes and surfactants. A water-insoluble salt refers herein to a salt which at a pH of 7 has a solubility in water of less than 5 g/l, preferably less than 3 g/l, especially less than 2 g/l and most preferably less than 1 g/I (at 25° C. and 1 bar). The use of a water-insoluble salt can reduce the tackiness due to the film-forming polymer, especially the polyurethane which appears in the course of heat-treating.

The water-insoluble salts are used as a solid material or in the form of dispersions, preferably as an aqueous dispersion. Solids are typically jetted into the apparatus as fine dusts by means of a carrier gas. The dispersion is preferably applied by means of a high speed stirrer by preparing the dispersion from solid material and water in a first step and introducing it in a second step rapidly into the fluidised bed preferably via a nozzle. Preferably both steps are carried out in the same apparatus. The aqueous dispersion can if appropriate be applied together with the polyurethane (or other film-forming polymer) or as a separate dispersion via separate nozzles at the same time as the polyurethane or at different times from the polyurethane. It is particularly preferable to apply the deagglomeration aid after the film-forming polymer has been applied and before the subsequent heat-treating step.

Suitable cations in the water-insoluble salt are for example Ca²⁺, Mg²⁺, A1³⁺, Sc³⁺, Y³⁺, Ln³⁺ (where Ln denotes lanthanoids), Ti⁴⁺, Zr⁴⁺, Li⁺, K⁺, Na⁺ or Zn²⁺. Suitable inorganic anionic counter ions are for example carbonate, sulphate, bicarbonate, orthophosphate, silicate, oxide or hydroxide. When a salt occurs in various crystal forms, all crystal forms of the salt shall be included. The water-insoluble inorganic salts are preferably selected from calcium sulphate, calcium carbonate, calcium phosphate, calcium silicate, calcium fluoride, apatite, magnesium phosphate, magnesium hydroxide, magnesium oxide, magnesium carbonate, dolomite, lithium carbonate, lithium phosphate, zinc oxide, zinc phosphate, oxides, hydroxides, carbonates and phosphates of the lanthanoids, sodium lanthanoid sulphate, scandium sulphate, yttrium sulphate, lanthanum sulphate, scandium hydroxide, scandium oxide, aluminium oxide, hydrated aluminium oxide and mixtures thereof. Apatite refers to fluoro apatite, hydroxyl apatite, chloro apatite, carbonate apatite and carbonate fluoro apatite. Of particular suitability are calcium and magnesium salts such as calcium carbonate, calcium phosphate, magnesium carbonate, calcium oxide, magnesium oxide, calcium sulphate and mixtures thereof. Amorphous or crystalline forms of aluminium oxide, titanium dioxide and silicon dioxide are also suitable. These deagglomeration aids can also be used in their hydrated forms. Useful deagglomeration aids further include many clays, talcum and zeolites. Silicon dioxide is preferably used in its amorphous form, for example as hydrophilic or hydrophobic Aerosil® (Evonik Degussa GmbH, Hanau, Germany), but selectively can also be used as aqueous commercially available silica sol, such as for example Levasil® silica sols (H. C. Starck GmbH, Goslar, Germany), which have particle sizes in the range 5-75 nm.

The average particle size of the finely divided water-insoluble salt is typically less than 200 μm, preferably less than 100 μm, especially less than 50 μm, more preferably less than 20 μm, even more preferably less than 10 μm and most preferably in the range of less than 5 μm. Fumed silicas are often used as even finer particles, e.g. less than 50 nm, preferably less than 30 nm, even more preferably less than 20 nm primary particle size.

In a preferred embodiment, the finely divided water-insoluble salt is used in an amount in the range from 0.001% to 20% by weight, preferably less than 10% by weight, especially in the range from 0.001% to 5% by weight, more preferably in the range from 0.001% to 2% by weight and most preferably between 0.001 and 1% by weight, based on the weight of the water-absorbing polymer.

In lieu of or in addition to the above inorganic salts it is also possible to use other known deagglomeration aids, examples being waxes and preferably micronised or preferably partially oxidised polyethylene waxes, which can likewise be used in the form of an aqueous dispersion. Such waxes are described in EP 0 755 964, which is hereby expressly incorporated herein by reference.

Furthermore, to achieve deagglomeration, a second coating with a dispersion of another polymer of high Tg (>50° C.) can be carried out.

Useful deagglomeration aids further include stearic acid, stearates—for example: magnesium stearate, calcium stearate, zinc stearate, aluminium stearate, and furthermore polyoxyethylene-20-sorbitan monolaurate and also polyethylene glycol 400 monostearate.

Useful deagglomeration aids likewise include surfactants. A surfactant can be used alone or mixed with one of the abovementioned deagglomeration aids, preferably a water-insoluble salt.

The addition can take place together with the polyurethane, before the addition of the polyurethane or after the addition of the polyurethane. In general, it can be added before heat-treating. The surfactant can further be applied during the post-crosslinking operation.

Useful surfactants include non-ionic, anionic and cationic surfactants and also mixtures thereof. The water-absorbing material preferably comprises non-ionic surfactants. Useful non-ionic surfactants include for example sorbitan esters, such as the mono-, di- or triesters of sorbitans with C₈-C₁₈-carboxylic acids such as lauric, palmitic, stearic and oleic acids; polysorbates; alkylpolyglucosides having 8 to 22 and preferably 10 to 18 carbon atoms in the alkyl chain and 1 to 20 and preferably 1.1 to 5 glucoside units; N alkylglucamides; alkylamine alkoxylates or alkylamide ethoxylates; alkoxylated C₈-C₂₂-alcohols such as fatty alcohol alkoxylates or oxo alcohol alkoxylates; block polymers of ethylene oxide, propylene oxide and/or butylene oxide; alkylphenol ethoxylates having C₆-C₁₄-alkyl chains and 5 to 30 mol of ethylene oxide units.

The amount of surfactant is generally in the range from 0.01% to 0.5% by weight, preferably less than 0.1% by weight and especially below 0.05% by weight, based on the weight of the water-absorbing material.

According to the invention, heat-treating takes place at temperatures above 50° C., preferably in a temperature range from 100 to 200° C., especially 120-160° C. Without wishing to be bound by theory, the heat-treating causes the applied film-forming polymer, preferably polyurethane, to flow and form a polymeric film whereby the polymer chains are entangled. The duration of the heat-treating is dependent on the heat-treating temperature chosen and the glass transition and melting temperatures of the film-forming polymer. In general, a heat-treating time in the range from 30 minutes to 120 minutes will be found to be sufficient. However, the desired formation of the polymeric film can also be achieved when heat-treating for less than 30 minutes, for example in a fluidised bed dryer. Longer times are possible, of course, but especially at higher temperatures can lead to damage in the polymeric film or to the water-absorbing material.

The heat-treating is carried out for example in a downstream fluidised bed dryer, a tunnel dryer, a tray dryer, a tower dryer, one or more heated screws or a disk dryer or a paddle dryer. Heat-treating is preferably done in a fluidised bed reactor and more preferably directly in the Wurster Coater.

The heat-treating can take place on trays in forced air ovens. In this case it is desirable to treat the coated polymer with a deagglomeration aid before heat-treating. Alternatively, the tray can be antistick-coated and the coated polymer then placed on the tray as a monoparticulate layer in order that sintering together may be avoided.

In one embodiment for the process steps of coating, heat-treating, and cooling, it may be possible to use air or dried air in each of these steps.

In other embodiments an inert gas may be used in one or more of these process steps. In yet another embodiment one can use mixtures of air and inert gas in one or more of these process steps.

The heat-treating is preferably carried out under inert gas. It is particularly preferable that the coating step be carried out under inert gas as well. It is very particularly preferable when the concluding cooling phase is carried out under protective gas too. Preference is therefore given to a process where the production of the water-absorbing material according to the present invention takes place under inert gas.

It is believed that the water-absorbing material obtained by the process according to the present invention is surrounded by a homogeneous film. Depending on the coating rate based on the absorbent polymeric particles and the way the application is carried out, the polymeric film may conceivably not be completely uninterrupted, but have uncovered areas, such as islands. This embodiment too is encompassed by the invention. A flawed, for example a coating with holes is not disadvantageous as long as the particles of the superabsorbent polymer are coated such that despite the flaws in the coating, substantially similar mechanical forces occur in the swelling of the coated water-absorbing polymeric particles as in the case of a substantially flawless coating. The hydrophilicity of the polymer plays a minor part for this embodiment. The deliberate incorporation of such imperfections e.g. via the use of fillers or polymeric additives to the dispersion may provide a means to increase the absorption speed of the claimed materials, and may be used as an advantage. It may be advantageous to include water soluble fillers in the coating that subsequently dissolve during the swelling of the coated water-absorbing material.

It is generally observed that flawless and flawed particles exist side by side, and this can be microscopically visualised by staining methods.

It may be advantageous in such cases that the absorbent polymeric particle is post-crosslinked, as detailed above. Already post-crosslinked water-absorbing polymeric particles can be coated with the film-forming polymer especially polyurethane. It is likewise possible for the post-crosslinker not to be applied until before heat-treating, i.e., concurrently with the film-forming polymer especially polyurethane in the fluidised bed or after the film-forming polymer-coating step. In the latter version of the process, this can be accomplished for example concurrently with the preferred addition of the deagglomeration aid. In all cases, heat-treating is preferably carried out at temperatures in the range from 120 to 160° C.

After the heat-treating step has been concluded, the dried water-absorbing polymeric materials are cooled. To this end, the warm and dry polymer is preferably continuously transferred into a downstream cooler. This can be for example a disk cooler, a Nara paddle cooler or a screw cooler. Cooling is via the walls and if appropriate the stirring elements of the cooler, through which a suitable cooling medium such as for example warm or cold water flows. Water or aqueous solutions or dispersions of additives may preferably be sprayed on in the cooler; this increases the efficiency of cooling (partial evaporation of water) and the residual moisture content in the finished product can be adjusted to a value in the range from 0% to 15% by weight, preferably in the range from 0.01% to 6% by weight and more preferably in the range from 0.1% to 3% by weight. The increased residual moisture content reduces the dust content of the product and helps to accelerate the swelling when such water-absorbing material is contacted with aqueous liquids. Examples for additives are triethanolamine, surfactants, silica, or aluminium sulphate.

Optionally, however, it is possible to use the cooler for cooling only and to carry out the addition of water and additives in a downstream separate mixer. Cooling lowers the product temperature only to such an extent that the product can easily be packed in plastic bags or within silo trucks. Product temperature after cooling is typically less than 90° C., preferably less than 60° C., most preferably less than 40° C. and preferably more than 20° C.

It may be preferable to use a fluidised bed cooler.

If coating and heat-treating are both carried out in fluidised beds, the two operations can be carried out either in separate apparatus or in one apparatus having communicating chambers. If cooling too is to be carried out in a fluidised bed cooler, it can be carried out in a separate apparatus or optionally combined with the other two steps in just one apparatus having a third reaction chamber. More reaction chambers are possible as it may be desired to carry out certain steps like the coating step in multiple chambers consecutively linked to each other, so that the water absorbing polymer particles consecutively build the film-forming polymer shell in each chamber by successively passing the particles through each chamber one after another.

Preference is given to process comprising the steps of
i) spraying the water-absorbing polymeric particles with a dispersion of an elastic film-forming polymer in a fluidised bed reactor at temperatures in the range from 0° C. to 50° C. preferably in the range from 0° C. to 45° C., and
ii) optionally coating the particles obtained according to a), with a deagglomeration aid and subsequently
iii) heat-treating the coated particles at a temperature above 50° C. and subsequently
iv) cooling the heat-treated particles to below 90° C.

The coated water-absorbing particles may be present in the water-absorbing material of the invention mixed with other particles components, such as fibres, (fibrous) glues, organic or inorganic filler materials or flowing aids, process aids, anti-caking agents, odour control agents, colouring agents, coatings to impart wet stickiness, hydrophilic surface coatings, etc.

It is possible that the water-absorbing material comprises two or more layers of coating agent (shells), obtainable by coating the water-absorbing polymers twice or more. This may be the same coating agent or a different coating agent. However, preference for economic reasons is given to a single coating with a film-forming polymer and preferably with polyurethane.

The water-absorbing material of the present invention is notable for the fact that the particles, which have an irregular shape when dry, assume in the swollen state a more rounded shape/morphology, since the swelling of the absorbent core is distributed via the rebound forces of the elastic polymeric envelope over the surface and the elastic polymeric envelope substantially retains its properties in this respect during the swelling process and in use. The enveloping film-forming polymer especially the polyurethane is permeable to saline, so that the polymer particles exhibit excellent absorption.

The water-absorbing material of the invention preferably comprises less than 20% by weight of water, or even less than 10% or even less than 8% or even less than 5%, or even no water. To improve the properties, the polymer particles can additionally be coated or re-moisturised.

The re-moisturizing is carried out preferably at 30 to 80° C., more preferably at 35 to 70° C. and most preferably at 40 to 60° C. At excessively low temperatures, the polymer particles tend to form lumps, and, at higher temperatures, water already evaporates noticeably. The amount of water used for re-moisturizing is preferably from 1 to 10% by weight, more preferably from 2 to 8% by weight and most preferably from 3 to 5% by weight. The re-moisturizing increases the mechanical stability and reduces the tendency to static charging.

Suitable coatings for improving the free swell rate (FSR) and the saline flow conductivity (SFC) are, for example, inorganic inert substances, such as water-insoluble metal salts, organic polymers, cationic polymers and di- or polyvalent metal cations, such as aluminium sulphate and aluminium lactate. Suitable coatings for dust binding are, for example, polyols. Suitable coatings for counteracting the undesired caking tendency of the polymer particles are, for example, fumed silica, such as Aerosil® 200, and surfactants, such as sorbitan monolaurate (“Span 20”). Suitable coatings for reducing the content of unconverted monomers (residual monomers) are, for example, reducing agents such as the salts of sulphurous acid, of hypophosphorous acid and/or of organic sulphinic acid. However, the reducing agent used is preferably a mixture of the sodium salt of 2-hydroxy-2-sulphinatoacetic acid, the disodium salt of 2-hydroxy-2-sulphonatoacetic acid and sodium hydrogen sulphite. Such mixtures are available as Brüggolite® FF6 and Brüggolite® FF7 (Bruggemann Chemicals; Heilbronn; Germany).

It may also be advantageous to re-moisturise and/or the coat at the stage of the polymeric foam prior to grinding to aid further processing.

The present invention further provides the water-absorbing polymer particles producible from foamed monomer solutions or suspensions by the process according to the invention.

The water-absorbing polymer particles finally produced by the process according to the invention preferably have a moisture content of 0 to 15% by weight, more preferably 0.2 to 10% by weight and most preferably 0.5 to 8% by weight.

The water-absorbing polymer particles produced by the process according to the invention have a centrifuge retention capacity (CRC) of typically at least 10 g/g, preferably at least 15 g/g, more preferably at least 20 g/g, especially preferably at least 22 g/g, very especially preferably at least 25 g/g. The centrifuge retention capacity (CRC) of the water-absorbing polymer particles is typically less than 40 g/g.

The water-absorbing polymer particles produced by the process according to the invention have an absorption under a pressure of 49.2 g/cm² (AUL 0.7 psi) of typically at least 10 g/g, preferably at least 13 g/g, more preferably at least 16 g/g, especially preferably at least 18 g/g, very especially preferably at least 20 g/g.

The water-absorbing polymer particles produced by the process according to the invention have a saline flow conductivity (SFC) of typically at least 5×10⁻⁷ cm³s/g, preferably at least 20×10⁻⁷ cm³s/g, more preferably at least 35×10⁻⁷ cm³s/g, most preferably at least 50×10⁻⁷ cm³s/g. The saline flow conductivity (SFC) of the water-absorbing polymer particles is typically less than 200×10⁻⁷ cm³s/g.

The process according to the invention leads to the water-absorbing polymer particles of the invention that exhibit high saline flow conductivity (SFC) and high free swell rate (FSR).

The inventive water-absorbing polymer particles may be mixed with non-inventive polymer gels and/or non-inventive water-absorbing polymer particles. The method of mixing is not subject to any restrictions. The proportion of the inventive water-absorbing polymer particles in the mixture is preferably from 0.1 to 90% by weight, more preferably from 1 to 50% by weight, most preferably from 5 to 25% by weight. The inventive mixtures are notable for surprisingly high saline flow conductivity (SFC).

The water-absorbing polymer particles of the present invention are useful in hygiene articles which comprise the inventive water-absorbing polymer particles. The hygiene articles typically comprise a water-impervious backside, a water-pervious topside and, in between, an absorbent core of the inventive polymer particles and cellulose fibres. The proportion of the inventive polymer particles in the absorbent core is preferably 20 to 100% by weight, more preferably 40 to 100% by weight, most preferably 60 to 100% by weight.

### Test Methods

The “WSP” test methods referred to below are described in “Standard Test Methods for the Nonwovens Industry”, 2005 edition, jointly published by “Worldwide Strategic Partners” EDANA (European Disposables and Nonwovens Association, Avenue Eugene Plasky, 157, 1030 Brussels, Belgium, www.edana.org) and INDA (Association of the Nonwoven Fabrics Industry, 1100 Crescent Green, Suite 115, Cary, N.C. 27518, U.S.A., www.inda.org). The publication is available from either EDANA or INDA.

The measurements should, unless stated otherwise, be carried out at an ambient temperature of 23±2° C. and a relative air humidity of 50±10%. The water-absorbing polymer particles are mixed thoroughly before the measurement.

### Centrifuge Retention Capacity (CRC)

Centrifuge Retention Capacity (CRC) is determined using Standard Test Method WSP 241.2 (05).

Absorption Under Load 0.3 psi (AUL 0.3 psi)

Absorption Under Load 0.3 psi (AUL 0.3 psi) (in SI units: absorption under a pressure of 21.0 g/cm²) is determined using Standard Test Method WSP 242.2 (05).

Absorption Under Load 0.7 psi (AUL 0.7 psi)

Absorption Under Load 0.7 psi (AUL 0.7 psi) (in SI units: absorption under a pressure of 49.2 g/cm²) is determined using Standard Test Method WSP 242.2 (05) with the following modification: A plastic piston and cylindrical weight with a total weight of 1347 grams instead of 527 grams are used to obtain a pressure of 0.7 psi.

### Particle Size Distribution (PSD)

Particle Size Distribution is determined using Standard Test Method WSP 220.2 (05).

### Mean Particle Size

The mean particle size of the product fraction is determined using Standard Test Method WSP 220.2 (05), where the proportions by mass of the screen fractions are plotted in cumulated form and the mean particle size is determined graphically. The mean particle size here is the value of the mesh size which gives rise to a cumulative 50% by weight.

### Water or Moisture Content

Water or Moisture Content is determined using Standard Test Method WSP 230.2 (05).

### Saline Flow Conductivity (SFC)

The saline flow conductivity (SFC) of a swollen gel layer under a pressure of 63.3 g/cm² (0.9 psi) is, as described in EP 0 640 330 A1, determined as the gel layer permeability of a swollen gel layer of water-absorbing polymer particles, the apparatus described on page 19 and in FIG. 8 in the aforementioned patent application having been modified to the effect that the glass frit (40) is not used, and the plunger (39) consists of the same polymer material as the cylinder (37) and now comprises 21 bores of equal size distributed homogeneously over the entire contact area. The procedure and evaluation of the measurement remain unchanged from EP 0 640 330 A1. The flow is detected automatically.

The saline flow conductivity (SFC) is calculated as follows:

SFC[cm³s/g]=(Fg(t=0)×L0)/(d×A×WP)

where Fg(t=0) is the flow of NaCl solution in g/s, which is obtained using linear regression analysis of the Fg(t) data of the flow determinations by extrapolation to t=0, L0 is the thickness of the gel layer in cm, d is the density of the NaCl solution in g/cm³, A is the area of the gel layer in cm², and WP is the hydrostatic pressure over the gel layer in dyn/cm².

### Free Swell Rate (FSR)

To determine the free swell rate (FSR), 1.00 g (=W1) of water-absorbing polymer particles are weighed into a 25 ml beaker and distributed homogeneously over the base thereof. Then 20 ml of a 0.9% by weight sodium chloride solution are metered into a second beaker and the contents of this beaker are added rapidly to the first, and a stopwatch is started. As soon as the last drop of the sodium chloride solution has been absorbed, which is evident by the disappearance of the reflection on the liquid surface, the stopwatch is stopped. The exact amount of liquid which has been poured out of the second beaker and absorbed by the water-absorbing polymer particles in the first beaker is determined accurately by reweighing the second beaker (=W2). The time required for the absorption, which was measured with the stopwatch, is designated as t. The disappearance of the last liquid drop on the surface is determined as the time t.

The free swell rate (FSR) is calculated therefrom as follows:

FSR[g/gs]=W2/(W1×t)

When the moisture content of the water-absorbing polymer particles is more than 3% by weight, the weight W1 has to be corrected by this moisture content.

### EXAMPLES

### Example 1

### Synthesis of Water-Absorbing Polymer Particles

In a glass flask of 5 I volume with a bottom outlet, the following amounts of components were continuously mixed:

| | | |
|---|---|---|
| 888.3 | g/h | acrylic acid, |
| 4636 | g/h | aqueous sodium acrylate solution, 37.3% by weight, |
| 4.35 | g/h | Irgacure ® 2959 [2-Hydroxy-4′-hydroxyethoxy-2- |
| | | methylpropiophenone, CAS-No: 106797-53-9] (BASF Schweiz |
| | | AG, Basel, Switzerland) |
| 91.4 | g/h | diacrylate ester of a polyethylene glycol having an average |
| | | molecular weight of 400 (“PEGDA-400”), |
| 180 | g/h | de-ionised water and |
| 209 | g/h | aqueous solution of Lutensol ® AT 80, 10% by weight |
| | | (ethoxylated alcohol, BASF SE, 67056 Ludwigshafen, |
| | | Germany). |

This mixture was transported via an excenter screw pump into a mixing tube and pressurised by admixing with

| | |
|---|---|
| 120 g/h | carbon dioxide and |
| 308 g/h | aqueous solution of “V50” [2, 2′-Azobis |
| | (2-methylpropionamidine dihydrochloride], |
| | 10% by weight. |

A pressure of 10 bar at an expansion valve placed after the mixing tube was kept constant. The monomer solution spontaneously turned into foam upon discharge from the expansion valve onto a polyester film moving at a speed of 1.0 m/min. A second polyester film was placed on top of the foam by means of a roller, setting the foam thickness to 1200 μm. The foam was irradiated by two 400 W, two 2000 W and another two 400 W ultraviolet lamps in sequence in a nitrogen-flushed reactor, resulting in a partly polymerised foam structure. The upper film was removed and the foam was irradiated by 28 ultraviolet tubes of 30 W each in a second nitrogenflushed reactor. Subsequently, the resulting foam polymer was sprayed with

| | |
|---|---|
| 4560 g/h | aqueous solution of sodium bicarbonate, 3% by weight |

and finally dried at 92° C. in a belt dryer of 8 m length to achieve a moisture content of 3 weight %.

The foam thus produced was pre-crushed to approx. 5×5 cm pieces. 100 g each of this material was filled into a kitchen machine (Braun Multiquick 7, 850 W, equipped with rotating blades, Braun GmbH, Frankfurter Str. 145, 61476 Kronberg/Taunus, Germany) and first milled 10 times for 1 s each at full power, then for 12 s also at full power. The product was then sifted with a sifting machine (Retsch AS200, amplitude 1.5 mm/′g′, Retsch GmbH, Retsch-Allee 1-5, 42781 Haan, Germany) with a lower sieve of 150 μm and an upper sieve of 850 μm for 5 min. The fraction of 150 μm-850 μm was collected.

The resulting polymer was designated Polymer 1.

### Example 2

### Surface Crosslinking

1800 g of Polymer 1 was placed in a cylindrical fluidised bed of 95 mm diameter having a gas distributor plate with holes of 2 mm diameter each, totaling 4.44% of the distributor plate area, and a central two-component nozzle. Fluidizing gas (26 m³/h nitrogen) was blown from the bottom through the distributor plate. The gas temperature was 20-25° C. and the fluidised bed was heat-traced at 35° C.

A surface crosslinker solution containing the following amounts of constituents was sprayed into the fluidised polymer powder at a rate of 5 g/min through the central nozzle, using 6 m³/h nitrogen to spray:

| | |
|---|---|
| 1.26 g | N-(2-hydroxyethyl)-2-oxazolidinone (“HEONON”) |
| 1.26 g | 1,3-propane diol |
| 10.5 g | 1,2-propane diol |
| 0.072 g | sorbitan monococoate (Emulsogen ® V4345, Clariant International |
| | Ltd., Rothausstrasse 61, 4132 Muttenz, Switzerland) |
| 16.344 g | 2-propanole |
| 38.124 g | water |

The resulting polymer was designated Polymer 2A.

Polymer 2A was placed in a pre-heated cylindrical fluidised bed of 150 mm diameter having a gas distributor plate with holes of 2 mm diameter each, totalling 3% of the distributor plate area, and heat-treated for 60 minutes. Fluidizing gas (23 m³/h nitrogen) was blown from the bottom through the distributor plate. The gas temperature was 195° C. and the fluidised bed was traceheated at 185-195° C.

The resulting polymer was designated Polymer 2B.

### Example 3

### Polymer Coating

Example 2 was repeated, however, directly after spraying the surface crosslinker solution, a polymer solution containing the following amounts of constituents was sprayed at a rate of 5 g/min:

| | |
|---|---|
| 94.737 g | aqueous dispersion of a polyurethane, 38% by weight |
| | (Astacin ® Finish PUMN TF, BASF SE, 67056 |
| | Ludwigshafen, Germany), and |
| 85.263 g | water |

followed, at a rate of 5 g/min, by

| | |
|---|---|
| 18.000 g | Levasil ® 50/50% (anionic colloidal silica sol, Kurt |
| | Obermeier GmbH & Co. KG, Berghäuser Straβe 70, |
| | 57319 Bad Berleburg, Germany), and |
| 27.000 g | water |

The resulting polymer was designated Polymer 3A.

Polymer 3A was placed in a pre-heated cylindrical fluidised bed of 150 mm diameter having a gas distributor plate with holes of 2 mm diameter each, totalling 3% of the distributor plate area, and heat-treated for 30 minutes. Fluidizing gas (23 m³/h nitrogen) was blown from the bottom through the distributor plate. The gas temperature was 195° C. and the fluidised bed was trace-heated at 185-195° C.

The resulting polymer was designated Polymer 3C. A sample taken after 20 min of heat-treatment was designated Polymer 3B.

### Example 4

### Polymer Coating

1500 g of Polymer 1 was placed in a cylindrical fluidised bed of 95 mm diameter having a gas distributor plate with holes of 2 mm diameter each, totalling 4.44% of the distributor plate area, and a central two-component nozzle. Fluidizing gas (26 m³/h nitrogen) was blown from the bottom through the distributor plate. The gas temperature was 20-25° C. and the fluidised bed was heat-traced at 35° C.

A polymer solution containing the following amounts of constituents was sprayed into the fluidised polymer powder at a rate of 5 g/min through the central nozzle, using 6 m³/h nitrogen to spray:

| | |
|---|---|
| 87.947 g | aqueous dispersion of a polyurethane, 38% by weight |
| | (Astacin ® Finish PUMN TF, BASF SE, 67056 |
| | Ludwigshafen, Germany), and |
| 71.053 g | water |

followed, at a rate of 5 g/min, by

| | |
|---|---|
| 15.000 g | Levasil ® 50/50% (anionic colloidal silica sol, Kurt |
| | Obermeier GmbH & Co. KG, Berghäuser Straβe 70, |
| | 57319 Bad-Berleburg, Germany), and |
| 15.000 g | water |

The resulting polymer was designated Polymer 4A.

Polymer 4A was placed in a pre-heated cylindrical fluidised bed of 150 mm diameter having a gas distributor plate with holes of 2 mm diameter each, totalling 3% of the distributor plate area, and heat-treated for 50 minutes. Fluidizing gas (23 m³/h nitrogen) was blown from the bottom through the distributor plate. The gas temperature was 195° C. and the fluidised bed was traceheated at 185-195° C.

The resulting polymer was designated Polymer 4C. A sample taken after 40 min of heattreatment was designated Polymer 4B.

The properties of the polymers (mean values of 2 (CRC, AUL 0.7 psi, FSR) resp. 3 (SFC) measurements) obtained are listed in the following table:

| Polymer | SFC | CRC | AUL 0.7 | FSR |
|---|---|---|---|---|
| (^{*)}comparative) | [10⁻⁷ cm³/s g] | [g/g] | psi [g/g] | [g/gs] |
| 1^{*)} | 39 | 17.1 | 17.0 | 1.26 |
| 2A^{*)} | 18 | 16.8 | 16.5 | 1.18 |
| 2B^{*)} | 136 | 17.1 | 18.2 | 0.88 |
| 3A^{*)} | 15 | 16.3 | 15.8 | 1.02 |
| 3B | 357 | 16.6 | 16.7 | 0.85 |
| 3C | 443 | 15.3 | 17.1 | 0.83 |
| 4A^{*)} | 28 | 16.4 | 16.7 | 1.22 |
| 4B | 385 | 16.0 | 15.4 | 0.85 |
| 4C | 496 | 15.6 | 15.7 | 0.79 |

The data demonstrate that the water-absorbing polymers of this invention exhibit superior permeability (very high SFC values), far more than can be achieved by surface post-crosslinking alone, and do so with our without surface post-crosslinking, without sacrificing absorption speed (FSR) or water absorption and retention properties (CRC and AUL values).

1. A process for producing water-absorbing polymer particles by polymerising a foamed aqueous monomer solution or suspension comprising
a) at least one ethylenically unsaturated monomer which bears an acid group and has been neutralised to an extent of 25 to 95 mol %,
b) at least one crosslinker,
c) at least one initiator,
d) optionally at least one surfactant,
e) optionally one or more ethylenically unsaturated monomer copolymerisable with the monomer mentioned under a),
f) optionally a solubiliser, and
g) optionally thickeners, foam stabilisers, polymerisation regulators, fillers, fibres and/or cell nucleators,
the monomer solution or suspension being polymerised to a polymeric foam that is dried, subsequently ground and classified, the process further comprising
i) spray-coating the water-absorbing polymeric particles with an elastic film-forming polymer in a fluidised bed reactor in the range from 0° C. to 50° C., and
ii) heat-treating the coated particles at a temperature above 50° C.

2. The process of claim 1, wherein step i) is performed continuously.

3. The process of claim 1, wherein step i) is performed by spraying the water-absorbing polymeric particles with a dispersion of the elastic film-forming polymer.

4. The process of claim 1, wherein the elastic film-forming polymer is a polyurethane.

5. The process according to claim 1, wherein at least 50 mol % of the neutralised monomer a) have been neutralised by an inorganic base.

6. The process according to claim 5, wherein the inorganic base is potassium carbonate, sodium carbonate, or sodium hydroxide.

7. The process according to claim 1, wherein the ground polymeric foam is classified to a particle size in the range from 100 to 1000 μm.

8. The process according to claim 1, wherein the monomer a) is acrylic acid.

9. Water-absorbing polymer particles obtainable by a process of claim 1.
